(19) United States
(12) Patent Application Publication
(10) Pub. No.: US 2007/0225255 A1
(43) Pub. Date: Sep. 27, 2007

(54) Use of Mitochondrially Targeted Antioxidant in the Treatment of Liver Diseases and Epithelial Cancers

(76) Inventors: Eleonore Frohlich, Graz (DE); Ivica Kvietikova, Graz (AT); Kurt Zatloukal, Graz (AT); Gottfried Schatz, Reinach (CH); Helmut Denk, Graz (AT); Cornelia Stumptner, Graz (AT); Charles Buck, West Lafayette, IN (US)

Correspondence Address: WENDEROTH, LIND & PONACK, L.L.P., 2033 K STREET N. W., SUITE 800, WASHINGTON, DC, 20006-1021, US

(21) Appl. No.: 11/632,149

(22) PCT Filed: Jul. 12, 2005
(86) PCT No.: PCT/EP05/53338

§ 371 Date: Feb. 12, 2007

(30) Foreign Application Priority Data
Jul. 13, 2004 (EP) 04103318.4

(51) Int. Cl. A61K 31/66

(52) U.S. Cl. 514/75

(57) ABSTRACT

The present invention relates to the use of a mitochondrially targeted antioxidant, e.g. derivatives of vitamin E, coenzyme Q₁₀ or a glutathione peroxidase mimetic, in the treatment and prevention of liver diseases and/or epithelial cancers. The present invention also relates to pharmaceutical compositions containing the antioxidant(s) intended for such use. Furthermore the invention relates to the manufacture of medicaments containing the antioxidant(s) useful for such prevention and treatment.

### TECHNICAL FIELD

The present invention relates to the use of a mitochondrially targeted antioxidant, e.g. derivatives of vitamin E, coenzyme Q₁₀ or a glutathione peroxidase mimetic, in the treatment and prevention of liver diseases and/or epithelial cancers.

### BACKGROUND ART

The spectrum of liver disease varies from mild and reversible fatty liver to progressive chronic liver disease, which results in the development of the life threatening conditions of liver cirrhosis, liver failure and liver cancer.

The major causes of chronic liver disease are infections with hepatitis B or C virus, excessive consumption of alcohol and non-alcoholic fatty liver disease (NAFLD).

Hepatitis B virus (HBV) infection is a global public health issue. It is the leading cause of cirrhosis and hepatocellular carcinoma (HCC) worldwide (Conjeevaram H. S. et al., 2003, Journal of Hepatology, 38: 90-103). Hepatitis C virus (HCV), a major cause of liver-related morbidity and mortality worldwide, represents one of the main public health problems (Alberti A. and Benvegnù L., Journal of Hepatology 2003, 38: 104-118). The HCV infection frequently causes chronic hepatitis, which is linked to the development of liver cirrhosis and HCC (Cyong J. C. et al., 2002, Am J Chin Med, 28: 351-360).

Alcoholic liver disease (ALD) is the commonest cause of cirrhosis in the Western world, currently one of the ten most common causes of death. In the United States, ALD affects at least 2 million people, or approximately 1% of the population. The true incidence of ALD, especially in its milder forms, may be substantially greater because many patients are asymptomatic and may never seek medical attention. The spectrum of ALD ranges from fatty liver (steatosis), present in most, if not all heavy drinkers, through steatohepatitis, cholestasis (characterised by blocked bile excretion from the liver), fibrosis and ultimately cirrhosis (Stewart S. F. and Day C. P, 2003, Journal of Hepatology, 38: 2-13). Although fatty liver is reversible with abstention, it is a risk factor for progression to fibrosis and cirrhosis in patients who continue drinking, particularly when steatohepatitis is present.

Non-alcoholic fatty liver disease (NAFLD) refers to a wide spectrum of liver damage, ranging from simple steatosis to steatohepatitis, cholestasis, advanced fibrosis and cirrhosis. Steatohepatitis (non-alcoholic steatohepatitis) represents only a stage within the spectrum of NAFLD (Anguilo P., 2002, N Engl. J. Med., 346: 1221-1231). The pathological picture resembles that of alcohol-induced liver injury, but it occurs in patients who do not abuse alcohol. NAFLD should be differentiated from steatosis, with or without hepatitis, resulting from secondary causes, because these conditions have distinctly different pathogens and outcomes. These secondary causes of fatty liver disease (steatosis) are nutritional (e.g. protein-calorie malnutrition, starvation, total parenteral nutrition, rapid weight loss, gastrointestinal surgery for obesity), drugs (e.g. glucocorticoids, synthetic estrogens, aspirin, calcium-channel blockers, tetracycline, valproic acid, cocaine, antiviral agents, fialuridine, interferon α, methotrexate, zidovudine), metabolic or genetic (e.g. lipodostrophy, dysbetalipoproteinemia, Weber-Christian disease, galactosaemia, glycogen storage disorders, acute fatty liver of pregnancy) and other, such as diabetes mellitus, obesity or hyperlipidaemia (Anguilo P., 2002, N Engl. J. Med., 346: 1221-1231; MacSween R. N. M. et al., 2002, Pathology of the Liver. Fourth Edition. Churchill Livingstone, Elsevier Science).

Despite the prevalence of chronic liver disorders effective therapies for most disorders in this category are absent.

A variety of inherited and acquired liver diseases are associated with alterations of the hepatocytic intermediate filament (IF) cytoskeleton. One of the most frequent IF-related alterations is the Mallory body (MB), which is formed in hepatocytes in alcoholic steato-hepatitis and non-alcoholic (ASH and NASH), chronic cholestasis, copper intoxication and other metabolic liver diseases as well as in some hepatocellular carcinomas (HCCs). MBs consist of aggregated misfolded keratin as major component as well as several proteins involved in the unfolded protein response (HSP27, HSP70, p62 and ubiquitin). Misfolding of proteins typically occurs as a consequence of protein modification in situations of cell stress, particularly oxidative stress. The chemical composition of MBs indicate that keratins are preferred targets for misfolding in stress situations and that MBs can be considered as a consequence of a cellular defense response to misfolded keratin (Denk et al., 2000, J. Hepatol., 32: 689-702).

The severest of the non-viral chronic liver diseases, alcoholic steatohepatitis and non-alcoholic steatohepatitis (ASH and NASH) lead with high frequency to liver cirrhosis, liver failure and liver cancer (e.g. HCC). ASH and NASH cannot be distinguished by morphologic evaluation in the diagnostic pathology laboratory. Increased fatty disposition accompanied by fibrosis, inflammation and alterations in liver cell (hepatocyte) morphology, however, indicate these more serious conditions. Cellular changes in ASH and NASH include increased size (ballooning) and presence of intracellular aggregates (e.g. MBs), and this spectrum of liver cell pathology is considered to be diagnostic for these conditions.

Overall, there is no proven specific treatment for ASH and NASH, having a definitive diagnosis via biopsy is not very likely to affect the management of the disease in a patient.

Although liver cancer is relatively uncommon in the industrialized western world, it is among the leading causes of cancer worldwide. In contrast to many other types of cancer, the number of people who develop and die from liver cancer is increasing.

On a global basis, primary liver cancer such as HCC belongs to the most common malignant tumors accounting for about 1 million deaths/year (Bruix, J. et al., 2004, Cancer Cell (5): 215-219).

The principal risk factors for liver cancer are viruses, alcohol consumption, food contamination with aflatoxin molds and metabolic disorders. The rates of alcoholism and chronic hepatitis B and C continue to increase. The outlook therefore is for a steady increase in liver cancer rates, underscoring the need for new therapies in this area.

Primary liver cancer is difficult to treat. Surgical removal of the cancer and liver transplantation is limited to small cancers and not a viable option for most patients since at diagnosis the cancer is often in an advanced stage. Chemotherapy is occasionally used for tumors not suitable for surgery but any benefit is usually short lived. Thus, survival rates for primary liver cancer are particularly low. Conventional therapy has generally not proven effective in the management of liver cancer.

For HCC for instance, there is no effective therapeutic option except resection and transplantation but these approaches are only applicable in early stages of HCC, limited by the access to donor livers, and associated with severe risks for the patient. In addition, these approaches are extremely expensive. These cancers respond very poorly to chemotherapeutics, most likely due to the normal liver function in detoxification and export of harmful compounds. Several other therapeutic options, such as chemoembolization, cryotherapy and ethanol injection are still in an experimental phase and the efficacy of these is not established.

Thus until now no satisfactory therapies have been developed in order to be able to intervene in liver disorders and other epithelial cancers.

It is already known that various antioxidants could be targeted to mitochondria by their covalent attachment to lipophilic cations by means of an alkylene chain (Smith R. A. J. et al., 1999, Eur. J. Biochem., 263: 709-716, and Kelso G. F. et al., 2001, J. Biol. Chem., 276: 4588-4596; James A. M. et al., 2005, J. Biol. Chem, 280: 21295-21312). This approach allows antioxidants to be targeted to a primary production site of free radicals and reactive oxygen species within the cell, rather than being randomly dispersed.

In particular, the targeting of vitamin E and coenzyme Q₁₀ derivatives (U.S. Pat. No. 6,331,532; WO 99/26954, WO2005/016322 and WO2005/016323) or a glutathione peroxidase mimetic (WO 2004/014927) to mitochondria by linking them to the triphenyl phosphonium ion has been described. Experiments in vitro showed that [2-(3,4-dihydro-6-hydroxy-2,5,7,8-tetramethyl-2H-1-benzopyran-2-yl)ethyl]-triphenylphosphonium bromide (MitoVit E) and a mixture of MitoQuino1 [10-(3,6-dihydroxy-4,5-dimethoxy-2-methylphenyl)decyl]triphenylphosphonium bromide and MitoQuinone [10-(4,5-dimethoxy-2-methyl-3,6-dioxo-1,4-cyclohexadien-1-yl)decyl]triphenylphosphonium bromide (MitoQ) (Kelso G. F. et al., loc. cit., and Smith R. A. J. et al., loc. cit. ) or MitoQ compound wherein anion is a methanesulfonate (James A. M. et al., 2005, J. Biol. Chem, 280: 21295-21312; WO2005/016322 and WO2005/016323) are rapidly and selectively accumulated by mitochondria within isolated cells.

In addition a mitochondria-targeted derivative of the spin trap of phenyl-t-butylnitrone (MitoPBN) has been developed (Smith R. A. J., Bioenergetics Group Colloquium, 2003, 679^{th} Meeting of the Biochemical Society: 1295-1299).

Importantly, the accumulation of these antioxidants by mitochondria protected them from oxidative damage far more effectively than untargeted antioxidants, suggesting that the accumulation of bioactive molecules within mitochondria does increase their efficacy while also decreasing harmful side reactions (Murphy M. P. and Smith R. A. J., 2000, Adv. Drug. Delivery Rev., 41: 235-250).

Furthermore, it was found that the simple alkyltriphenylphosphonium cation TPMP, MitoVit E and MitoQ could be fed safely to mice on a long term basis, generating potentially therapeutically effective concentrations within the brain, heart, liver, and muscle (Smith R. A. et al., 2003, PNAS, 100(9): 5407-5412).

The industrial application of these compounds (U.S. Pat. No. 6,331,532, WO 99/26954 or WO 2004/014927, WO2005/016322 and WO2005/016323) was claimed for use in preventing the elevated mitochondrial oxidative stress associated with neurodegenerative diseases, such as Parkinson's disease, Friedrich's Ataxia, Wilson's disease, diseases associated with mitochondrial DNA mutations, diabetes, motor neuron disease, inflammation and ischemic reperfusion tissue injury in strokes, heart attacks, organ transplantation and surgery, and the non-specific loss of vigour associated with ageing. In addition use of these compounds as prophylactics to protect organs during transplantation, to ameliorate the ischemic reperfusion injury that occurs during surgery, to reduce cell damage following stroke and heart attack, or as prophylactics given to premature babies, who are susceptible to brain ischemia, has been claimed in the mentioned patent documents.

Interest in the potential value of antioxidant therapy in the treatment of alcoholic hepatitis (AH) has arisen as a result of the growing body of evidence implicating oxidative stress as a key mechanism in alcohol-mediated hepatotoxicity (Stewart S. F. and Day C. P., 2003, Journal of Hepatology, 38: 2-13). These considerations have recently led to several trials investigating the effect of antioxidant supplementation in patients with severe AH (e.g. Philips M. et al., 2001, Journal of Hepatology, 34: 250A). In the most recent study (Stewart S. F. et al., 2002, Journal of Hepatolology, 36:16) the active group received a loading dose of N-acetylcysteine 150 mg/kg followed by 100 mg/kg/day for 1 week, and vitamins A-E, biotin, selenium, zinc, manganese, copper, magnesium, folic acid and coenzyme Q daily for 6 months. This antioxidant therapy showed no benefit either alone or in combination with steroids. In summary, on the basis of the data available thus far, high dose anti-oxidant therapy confers no survival benefit in patients with severe AH (Stewart S. F. and Day C. P., loc. cit.).

Oxidative stress has been implicated also in the pathogenesis of non-alcoholic fatty liver disease (NAFLD). In the study with choline deficient diet fed rats, vitamin E known to react with reactive oxygen species (ROS) by blocking the propagation of radical reactions in wide range of oxidative situations, however, neither prevented the development of fatty liver nor reduced the oxidative stress (Oliveira C. P. et al., 2003, Nutr. J., 2(1): 9).

In studies with patients having liver cirrhosis and a history of hepatitis C virus (HCV) infection treated by alpha-tocopherol (VitE group), there has been shown neither improvement of liver function, suppression of hepatocarcinogenesis, nor improvement of cumulative survival (Tagaki H. et al., 2003, Int. J. Vitam Nutr. Res., 73(6): 411-5).

Furthermore, in a randomized, multicentre study of 120 consecutive patients affected by biopsy-proven chronic hepatitis C who had been non responders to a previous course of alpha-interferon, oral supplementation with N-acetyl cysteine (1200 mg/day) and vitamin E (600 mg/day) did not improve the poor efficacy of re-treatment with alpha-interferon alone (Ideo, G., et al., 1999, Eur. J. Gastroenterol. Hepatol., 11 (11): 1203-7).

### SUMMARY OF THE INVENTION

The invention relates to the use of a mitochondrially targeted antioxidant compound comprising a lipophilic cation covalently coupled to an antioxidant moiety for the treatment or prophylaxis of liver diseases and/or epithelial cancers.

### DETAILED DESCRIPTION

It has now unexpectedly been found that the use of mitochondrially targeted antioxidants, e.g. derivatives of vitamin E, coenzyme Q₁₀ or glutathione peroxide mimetic, is useful in the treatment and prevention of liver diseases and/or epithelial cancers.

In its broadest aspect, the invention provides a mitochondrially targeted antioxidant which comprises a lipophilic cation covalently coupled to an antioxidant moiety, wherein the antioxidant moiety is capable of being transported through the mitochondrial membrane and accumulated within the mitochondria of intact cells, for use in the treatment and prevention of liver diseases and/or epithelial cancers. In particular, the compound according to invention prevents cellular damage resulting from oxidative stress (or free radicals) in the mitochondria.

The term “liver disease” according to invention refers to and comprises all kinds of disorders that affect the anatomy, physiology, metabolism, and/or genetic activities of the liver, that affect the generation of new liver cells and/or the regeneration of the liver, as a whole or parts thereof, transiently, temporarily, chronically or permanently, in a pathological way.

In particular, included are liver diseases caused by alcohol (e.g. ASH), non-alcoholic fatty liver changes (such as NAFLD including NASH), nutrition-mediated liver injury (for example starvation), other toxic liver injury (such as unspecific hepatitis induced by e.g. drugs such as but not limited to acetaminophen (paracetamol), chlorinated hydrocarbons (e.g. CCl₄), amiodarone (cordarone), valproate, tetracycline (only i.v.), isoniacid (Drug-induced liver disease 2004. Lazerow S K, Abdi M S, Lewis J H. Curr Opin Gastroenterol., 2005, 21(3): 283-292), or food intoxication resulting in acute or chronic liver failure, e.g. by consumption of mushrooms containing aflatoxins (preferably B1 aflatoxin) or ingestion of certain metal (such as copper or cadmium) or herbal products used in natural medicine (homeopoatics such as Milk thistle, Chaparral, Kawa-Kawa), interference of bilirubin metabolism, hepatitis like syndromes, cholestasis, granulomatous lesions, intrahepatic vascular lesions and cirrhosis), trauma and surgery (e.g. Pringle maneuver), radiation-mediated liver injury (such as caused by radiotherapy).

Liver disease is further understood to comprise infectious liver disease [caused e.g. by hepatitis B virus (HBV) and hepatitis C virus (HCV) infections] and autoimmune-mediated liver disease (e.g. autoimmune hepatitis). Further included is liver injury due to sepsis.

Liver disease is further understood to comprise genetic liver disorders (such as heamo-chromatosis and alphal antitrypsin deficiency), and other inherited metabolic liver diseases [e.g. metabolic steatohepatitis (MSH)].

Preferred examples of liver disorders to be treated include alcoholic liver disease (ALD), non-alcoholic fatty liver disease (NAFLD), steatosis, cholestasis, cirrhosis, acute and chronic hepatitis, heamochromatosis and alphal antitrypsin deficiency.

Within the meaning of the present invention the term “liver disease” according to invention also encompasses tumors (primary liver neoplasia) and tumor like lesions of the liver (such as focal nodular hyperplasia, FNH).

Liver disease is further understood to comprise liver neoplastic diseases such as benign liver neoplasms (e.g. liver cell adenoma) as well as liver cancer, for example hepatocellular carcinoma (HCC). HCC further comprises subtypes of the mentioned disorders, including liver cancers characterized by intracellular proteinaceous inclusion bodies, HCCs characterized by hepatocyte steatosis, and fibrolamellar HCC. For example, precancerous lesions are also included such as those characterized by increased hepatocyte cell size (the “large cell” change), and those characterized by decreased hepatocyte cell size (the “small cell” change) as well as macro regenerative (hyperplastic) nodules (Anthony P. in MacSween et al., eds. 2001, Pathology of the Liver, Churchill Livingstone, Edinburgh, UK).

The term “epithelial cancer” within the meaning of the invention includes carcinomas of organs other than liver, selected from the group consisting of lung, kidney, pancreas, prostate, skin and breast, and of gastrointestinal system such as stomach, kidney, and colon. The term “epithelial cancer” according to the invention refers to disorders of these organs in which epithelial cell components of the tissue are transformed resulting in a malignant tumor identified according to the standard diagnostic procedures as generally known to a person skilled in the art.

A preferred embodiment represents the use of the mitochondrially targeted antioxidant compound comprising a lipophilic cation covalently coupled to an antioxidant moiety in the treatment and prevention of liver disease, wherein the liver disease is a disease selected from the group consisting of alcoholic liver disease, non-alcoholic fatty liver disease, steatosis, cholestasis, liver cirrhosis, nutrition-mediated liver injury, toxic liver injury, infectious liver disease, liver injury in sepsis, autoimmune-mediated liver disease, hemochromatosis, alphal antitrypsin deficiency, radiation-mediated liver injury, liver cancer, benign liver neoplasms and focal nodular hyperplasia.

Another preferred embodiment represents the use of the mitochondrially targeted antioxidant compound comprising a lipophilic cation covalently coupled to an antioxidant moiety in the treatment and prevention of liver disease, wherein the liver disease is a disease selected from the group consisting of alcoholic liver disease, non-alcoholic fatty liver disease, steatosis, cholestasis, liver cirrhosis, nutrition-mediated liver injury, toxic liver injury, infectious liver disease, liver injury in sepsis, autoimmune-mediated liver disease, hemochromatosis, alphal antitrypsin deficiency, radiation-mediated liver injury.

The invention relates to the use of a mitochondrially targeted antioxidant compound comprising a lipophilic cation covalently coupled to an antioxidant moiety in the preparation of a medicament for the treatment or prophylaxis of liver diseases and epithelial cancers.

A preferred embodiment represents the use of the mitochondrially targeted antioxidant according to the invention in the preparation of a medicament for the treatment or prevention of liver disease, wherein the liver disease is a disease selected from the group consisting of alcoholic liver disease, non-alcoholic fatty liver disease, steatosis, cholestasis, liver cirrhosis, nutrition-mediated liver injury, toxic liver injury, infectious liver disease, liver injury in sepsis, autoimmune-mediated liver disease, hemochromatosis, alphal antitrypsin deficiency, radiation-mediated liver injury, liver cancer, benign liver neoplasms and focal nodular hyperplasia.

Yet another preferred embodiment is the use of the mitochondrially targeted antioxidant according to the invention in the preparation of a medicament for the treatment or prevention of liver disease, wherein the liver disease is a disease selected from the group consisting of alcoholic liver disease, non-alcoholic fatty liver disease, steatosis, cholestasis, liver cirrhosis, nutrition-mediated liver injury, toxic liver injury, infectious liver disease, liver injury in sepsis, autoimmune-mediated liver disease, hemochromatosis, alphal antitrypsin deficiency, radiation-mediated liver injury.

Another preferred embodiment is the use of the mitochondrially targeted antioxidant compound according to invention wherein the liver disease is alcoholic liver disease or non-alcoholic fatty liver disease.

A further preferred embodiment represents the use of the mitochondrially targeted antioxidant compound according to invention wherein the liver disease is alcoholic steatohepatitis or non-alcoholic steatohepatitis.

Another preferred embodiment is the use of the mitochondrially targeted antioxidant compound according to invention wherein the liver disease is alcoholic steatohepatitis.

Yet another preferred embodiment is the use of the mitochondrially targeted antioxidant compound according to invention wherein the liver disease is non-alcoholic steatohepatitis.

Within the meaning of the invention the term “disease according to invention” encompasses liver disorders and epithelial cancers as defined above.

A preferred embodiment represents the use of the mitochondrially targeted antioxidant compound for the treatment or prophylaxis of a disease according to invention wherein the liphophilic cation is the triphenylphosphonium cation.

Other lipophilic cations which may covalently be coupled to antioxidants in accordance with the present invention include the tribenzyl or triphenyl ammonium cation or the tribenzyl or a substituted triphenyl phosphonium cation.

In another preferred embodiment said mitochondrially targeted compound according to invention has the formula P(Ph)₃⁺XR.Z⁻ wherein X is a linking group, Z is an anion and R is an antioxidant moiety and the lipophilic cation represents the triphenylphosphonium cation, as shown by the general formula

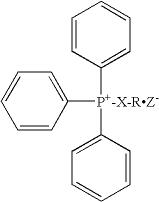

X as a linking group may be a carbon chain, one or more carbon rings, or a combination thereof, and such chains or rings wherein one or more carbon atoms are replaced by oxygen (forming ethers or esters) and/or by nitrogen (forming amines or amides).

While it is generally preferred that the carbon chain is an alkylene group, carbon chains which include one or more double or triple bonds are also within the scope of the invention. Also included are carbon chains carrying one or more substituents (such as oxo, hydroxyl, carboxylic acid or carboxamide groups), and/or one or more side chains or branches selected from unsubstituted or substituted alkyl, alkenyl or alkynyl groups.

Preferably, X is a C₁-C₃₀, more preferably C₁-C₂₀, most preferably C₁-C₁₅ carbon chain.

Preferably, X is (CH₂)ₙ, wherein n is an integer from 1 to 20, more preferably from about 1 to about 15.

In some particularly preferred embodiments, the linking group X is an ethylene, propylene, butylene, pentylene or decylene group.

In one particularly preferred embodiment the antioxidant moiety R is a quinone. In another preferred embodiment the antioxidant R moiety is a quinol. A quinone and corresponding quinol are equivalents since they are transformed to each other by reduction and oxidation, respectively.

In other embodiment the antioxidant moiety R is selected from the group consisting of vitamin E and vitamin E derivatives, chain breaking antioxidants, including butylated hydroxyanisole, butylated hydroxytoulene, general radical scavengers including derivatised fullerenes, spin traps including derivatives of 5,5-methylpyrroline N-oxide, tert-butylnitrosobenzene, α-phenyl-tert-butylnitrone and related compounds.

In a further preferred embodiment the antioxidant moiety R is vitamin E or a vitamin E derivative.

In another preferred embodiment the antioxidant moiety R is butylated hydroxyanisole or butylated hydroxytoulene.

In still further preferred embodiment the antioxidant moiety R represents a derivatised fullerene.

In some particularly preferred embodiments the antioxidant moiety R is a 5,5-dimethylpyrroline N-oxide, tert-butylnitrosobenzene, α-phenyl-tert-butylnitrone and derivatives thereof.

Preferably, Z⁻ is a pharmaceutically acceptable anion. Such pharmaceutically acceptable anions are formed from organic or inorganic acids. Suitable inorganic acids are, for example, halogen acids, such as hydrochloric acid, hydrobromic acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, alkane sulfonic acid such as methane- or ethane-sulfonic acid, 2-hydroxyethanesulfonic acid, ethane-1,2-disulfonic acid, arylsulfonic acid such as benzenesulfonic acid, 2-naphthalenesulfonic acid, 1,5-naphthalene-disulfonic acid or 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In one preferred embodiment Z⁻ is halide. In another preferred embodiment Z⁻ is bromide.

In a further preferred embodiment Z⁻ is the anion of an alkane- or arylsulfonic acid. In one particularly preferred embodiment Z⁻ is methanesulfonate.

In another particularly preferred embodiment, the mitochondrially targeted antioxidant useful in the treatment and prevention of liver diseases and/or epithelial cancers has the formula 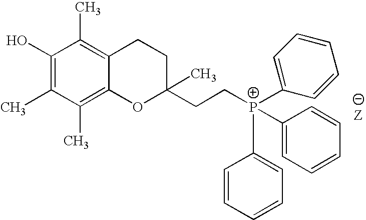
including all stereoisomers thereof wherein Z⁻ is a pharmaceutically acceptable anion, preferably Br⁻. This compound is referred to herein as “MitoVit B”.

In another preferred embodiment, the mitochondrially targeted antioxidant useful in the treatment and prevention of diseases according to the invention has the general formula 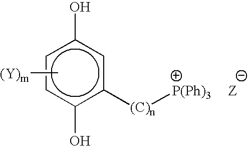
wherein Z⁻ is a pharmaceutically acceptable anion, preferably a halogen, m is an integer from 0 to 3, each Y is independently selected from groups, chains and aliphatic and aromatic rings having electron donating and accepting properties, (C)ₙ represents a carbon chain optionally carrying one or more double or triple bonds and optionally including one or more substituents and/or unsubstituted or substituted alkyl, alkenyl or alkynyl side chains, and n is an integer from 1 to 20.

Preferably, each Y is independently selected from the group consisting of alkoxy, alkylthio, alkyl haloalkyl, halo, amino, nitro, optionally substituted aryl, or when m is 2 or 3, two Y groups, together with the carbon atoms to which they are attached, form an aliphatic or aromatic carbocyclic or heterocyclic ring fused to the aryl ring. More preferably, each Y is independently selected from methoxy and methyl.

Preferably, (C)ₙ is an alkyl chain of the formula (CH₂)ₙ.

In a particularly preferred embodiment, the mitochondrially targeted antioxidant according to the invention has the formula 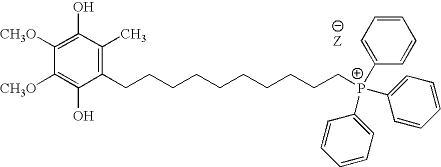
wherein Z⁻ is a pharmaceutically acceptable anion, preferably Br⁻ referred to herein as “MitoQuino1”, or an oxidized form of the compound (wherein the hydroquinone of the formula is a quinone) referred to herein as “MitoQuinone”. A mixture of varying amounts of MitoQuino1 and MitoQuinone is referred to as “MitoQ”.

Even more preferably, the mitochondrially targeted antioxidant according to the invention has the formula 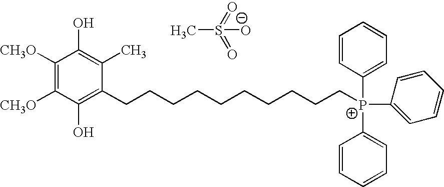
wherein the pharmaceutically acceptable anion Z⁻ is methanesulfonate. In this embodiment a mixture of varying amounts of MitoQuino1 and MitoQuinone is referred to as “MitoS”.

Further preferred embodiment according to invention represents the mitochondrially targeted derivative of the spin trap phenyl-t-butylnitrone of the following formula 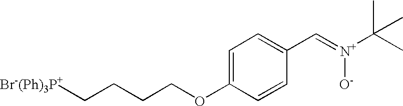
referred to herein as “MitoPBN”.

In another embodiment according to the invention the mitochondrially targeted antioxidant is a glutathione peroxidase mimetic such as a selenoorganic compound, i.e. an organic compound comprising at least one selenium atom. Preferred classes of selenoorganic glutathione peroxidase mimetics include benzisoselenazolones, diaryl diselenides and diaryl selenides.

In particular the glutathione peroxidase mimetic moiety is 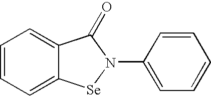
referred to herein as “Ebelsen” (2-phenyl-benzo[d]isoselenazol-3-one).

Preferred compounds of the invention have the formula 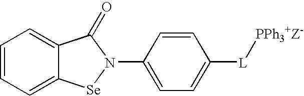
wherein Z⁻ is a pharmaceutically acceptable anion, preferably Br⁻ and L is a monosaccharide.

One particularly preferred embodiment according to invention has the formula 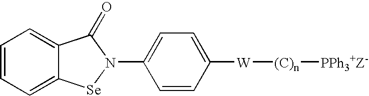 wherein Z⁻ and (C)n are defined as above, W is O, S or NH, preferably O or S, and n is from 1 to 20, more preferably 3 to 6.

In a further aspect, the present invention provides a pharmaceutical composition suitable for treatment and/or prophylaxis of a patient suffering from liver disease and/or epithelial cancer, which comprises an effective amount of a mitochondrially targeted antioxidant according to the present invention in combination with one or more pharmaceutically acceptable carriers or diluents, such as, for example, physiological saline solution, demineralized water, stabilizers (such as β-cyclodextrin, preferably in ratio 1:2), and/or proteinase inhibitors.

The term “pharmaceutically acceptable” as used herein pertains to compounds, ingredients, materials, compositions, dosage, forms etc., which are within the scope of sound medical judgment, suitable for use in contact with the tissues of the subject in question (preferably human) without excessive toxicity, irritation, allergic response, or other problem or complication, commensurate with a reasonable benefit/risk ratio. Each carrier, diluent, excipient etc. must also be “acceptable” in the sense of being compatible with the other ingredients of the formulation.

In still a further aspect, the invention provides a method of therapy or prophylaxis of a patient suffering from liver disease and/or epithelial cancer who would benefit from reduced oxidative stress, which comprises the step of administering to said patient a mitochondrially targeted antioxidant as defined above.

The term “treatment” within the meaning of the invention refers to a treatment that preferably cures the patient from at least one disorder according to the invention and/or that improves the pathological condition of the patient with respect to one or more symptoms associated with the disorder, on a transient, short-term (in the order of hours to days), long-term (in the order of weeks, months or years) or permanent basis, wherein the improvement of the pathological condition may be constant, increasing, decreasing, continuously changing or oscillatory in magnitude as long as the overall effect is a significant improvement of the symptoms compared with a control patient.

Further, the term “treatment” as used herein in the context of treating liver diseases and/or epithelial cancers pertains generally to treatment and therapy of a human or an animal (e.g., in veterinary applications), in which some desired therapeutic effect is achieved, for example the inhibition of the progress of the condition, and includes a reduction in the rate of progress, a halt in the rate of progress, amelioration of the condition, and cure of the condition.

The term “treatment” according the invention includes combination treatments and therapies, in which two or more treatments or therapies are combined, for example sequentially or simultaneously. Treatment as a prophylactic measure (i.e. prophylaxis) is also included.

Treatment according to the invention can be carried out in a conventional manner generally known to the person skilled in the art, e.g. by means of oral application or via intravenous injection of the pharmaceutical compositions according to the invention.

Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions that exhibit large therapeutic indexes are preferred. The dose must be adjusted to the age, weight and condition of the individual patient to be treated, as well as the route of administration, dosage form and regimen, and the result desired, and the exact dosage should of course be determined by the practitioner.

The actual dosage depends on the nature and severity of the disorder being treated, and is within the discretion of the physician, and may be varied by titration of the dosage to the particular circumstances of this invention to produce the desired therapeutic effect. However, it is presently contemplated, that pharmaceutical compositions comprising of from about 0.1 to 500 mg/kg of the active ingredient per individual dose, preferably of from about 0.1 to 100 mg/kg, most preferred from about 0.1 to 10 mg/kg, are suitable for therapeutic treatments.

In general, a suitable dose of the active compound according to invention is in the range of about 0.1 mg to about 250 mg per kilogram body weight of the subject to be treated per day.

The active ingredient may be administered in one or several dosages per day. A satisfactory result can, in certain instances, be obtained at a dosage as low as 0.1 mg/kg intravenously (i.v.) and 1 mg/kg per orally (p.o.). Preferred ranges are from 0.1 mg/kg/day to about 10 mg/kg/day i.v. and from 1 mg/kg/day to about 100 mg/kg/day p.o.

Furthermore the invention relates to the manufacture of medicaments containing the antioxidant compounds according to invention useful in the treatment and/or prevention of liver diseases and/or epithelial cancers, using standard procedures known in the prior art of mixing or dissolving the active compound with suitable pharmaceutical carriers. Such methods include the step of bringing into association the active compound with a carrier which comprises one or more accessory ingredients. In general the formulations according to invention are prepared by uniformly and intimately bringing into association the active compound with carriers (e.g. liquid carriers, finely divided solid carrier) and then shaping the product, if necessary. Suitable carriers, diluents and excipients used in the present invention can be found in standard pharmaceutical texts (see for example Handbook for Pharmaceutical Additives, 2001, 2^{nd} edition, eds. M. Ash and I. Ash).

The antioxidant compounds according to the invention e.g. derivatives of vitamin E, coenzyme Q₁₀ or a glutathione peroxidase mimetic, may be synthesized according to any of the known processes for making those compounds described in e.g. U.S. Pat. No. 6,331,532, WO 99/26954, WO 2004/014927 or WO 2003/016323).

It will be apparent to those skilled in the art that various modifications can be made to the compositions, methods and processes of this invention. Thus, it is intended that the present invention cover such modifications and variations, provided they come within the scope of the appended claims and their equivalents. All publications cited herein are incorporated in their entireties by reference.

To practically assess the impact of mitochondrially targeted antioxidants, e.g. derivatives of vitamin E, coenzyme Q₁₀ or a glutathione peroxidase mimetic, in the treatment and/or prevention of liver diseases according to the invention, the presence of morphological alterations such as inflammatory cells around the portal vein (Glisson's trias) and the degree of hepatocyte damage (necrosis, collapse of cytoskeleton (Example 3, FIG. 1), including but not limited to ballooning of hepatocytes, formation of a denser keratin intermediate filament (IF) network, reduced density of the keratin IF, and presence of Mallory bodies (MBs) representing one of the most frequent IF-related cytoskeleton alterations in various inherited and acquired liver diseases, with or without treatment with these antioxidants is evaluated (Examples 2 and 3).

The morphological alterations including MBs can be reproduced in mice by chronic intoxication with the fungistatic antimicrotubular drug griseofulvin (GF) or porphyrogenic agent 3,5-diethoxycarbonyl-1,4-dihydrocollidine (DDC) (Denk H. et al., 1975, Lab. Invest.: 773-776; Tsunoo C. et al., 1987, J. Hepatol., 5: 85-97). MBs formation can be induced in mouse livers by feeding a DDC- or GF containing diet (see Example 1). It is assumed that the oxidative injury induced by the methyl radical is the common pathogenetic principle in DCC or GF-fed animals and human livers with ASH or NASH, where free radicals produced by cytochrome P450-mediated oxidation of ethanol as well as the mitochondrial injury caused by acetaldehyde and free fatty acid overload are central features (Lieber C. S., 2000, J. Hepatol., 32: 113-128; Anguilo P., 2002, N Engl. J. Med., 346: 1221-1231).

Furthermore, it is widely accepted that in DDC- or GF fed mice the alterations of the IF keratin cytoskeleton as well as structure and chemical composition of MBs are very similar, if not identical, to the alterations found in human ASH and NASH (Denk H. et al., 2000, J. Hepatol., 32: 689-702). In this context it is noteworthy that other mouse models for alcoholic liver disease based on feeding alcohol-containing diets reproduce the disturbance of fat metabolism and, to some degree, inflammation of human ASH but not the alterations of the keratin IF cytoskeleton and do not lead to MB formation.

In one type of experiment (Example 2) the appearance of large MBs typically located in the perinuclear cytoplasmic region is detected in tested mice upon 6 to 10 weeks of intoxication using routine immunohistochemistry (such as heamotoxylin & eaosin staining) or immunofluorescence microscopy standard methods e.g. with the antibody SMI 31 directed against p62 protein (Zatloukal K. et al., 2002, Am J Pathol. 160(1):255-63). P62 has been originally identified as a phosphotyrosine-independent ligand of the SH2 domain of p56^{lck}, and as a cytoplasmic non-proteasomal ubiquitin-binding protein (Vadlamudi R. K. et al., 1996, J. Biol. Chem., 271: 20235-20237). A general role of p62 in the cellular stress response is implied since p62 expression is increased by a variety of stress stimuli, particularly oxidative stress (Ishii T. et al., 1996, Biochem Biophys. Res Comm., 226: 456-460).

At 4 weeks of recovery from intoxication, there are groups of hepatocytes devoid of cytoplasmic keratin filaments but still containing small remnants of MBs at the cell periphery in association with desmosomes. If mice are reexposed to GF or DDC, numerous MBs reappear within 24 to 72 hours (Stumptner C. et al., 2001, J. Hepatol., 34: 665-675). This enhanced formation of MBs upon reintoxication was interpreted—in analogy to allergic reactions—as a toxic memory effect.

To evaluate the impact of the antioxidants according to the invention on regression of morphological alterations in early stages of DDC- or GF intoxicated mice livers a positive control group of animals (3 to 7 days exposure to GF or DDC only) is compared to DDC- or GF intoxicated mice treated for further 3 to 7 days with e.g. MitoQ (a mixture of MitoQuino1 [10-(3,6-dihydroxy-4,5-dimethoxy-2 methylphenyl)decyl]triphenylphosphonium bromide and MitoQuinone [10-(4,5-dimethoxy-2-methyl-3,6-dioxo-1,4-cyclohexadien-1-yl)decyl]triphenylphosphonium bromide) or MitoVit E [2-(3,4-dihydro-6-hydroxy-2,5,7,8-tetramethyl-2H-1-benzopyran-2-yl)ethyl]-triphenylphosphonium bromide), respectively. Tested mice receive intraperitoneal (i.p.) or intravenous (i.v.) (tail vein) injections comprising the antioxidant compounds according to the invention, e.g. Mito Q or MitoVit E, and these mice are compared with vehicle-injected control mice (PBS supplemented with sufficient DMSO to maintain solubility of antioxidants) and other appropriate control mice (Example 3).

Furthermore, MitoQ or MitoQ derivatives such as MitoS (a mixture of MitoQuino1 [10-(3,6-dihydroxy-4,5-dimethoxy-2 methylphenyl)decyl]triphenylphosphonium methane sulfonate and MitoQuinone [10-(4,5-dimethoxy-2-methyl-3,6-dioxo-1,4-cyclohexadien-1-yl)decyl]triphenylphosphonium methane sulfonate or MitoVit E is supplemented to the diet. Doses are determined by measuring water or liquid diet consumption and mouse weight (Smith R. A. J. et al., 2003, PNAS, 100(9): 5407-5412).

In a further type of experiment a group of DDC- or GF intoxicated animals is simultaneously treated with antioxidant(s) according to the invention (e.g. Mito Q or MitoS) for 3 to 7 days and are then compared to a control group exposed for 3 to 7 days to DDC or GF only (Example 3).

In another set of experiments 3 to 12 mg/kg of MitoQ is simultaneously given intraperitoneally to DDC intoxicated mice for 3 days and compared to control animals. To practically assess in these short-term experiments the impact of mitochondrially targeted antioxidants according to the invention (e.g. MitoQ or MitoS) the presence (or absence) of inflammatory cells around the portal vein (Glisson's trias) and the degree of hepatocyte damage such as necrosis, collapse of cytoskeleton (see FIGS. 2 and 3) instead of cell ballooning and/or Mallory bodies analysis (typical for long term exposure to DDC or GF, respectively) are compared to the appropriate controls (FIGS. 1 to 3). Both cell ballooning and Mallory bodies are not suited in these experiments due to the fact that they are not formed within this short time exposure to DDC to a degree that allows statistical evaluations.

Overall, under MitoQ treatment the normal architecture represented by strands of hepatocytes bordered by sinusoids is again visible. The morphology of the hepatocytes is normal regarding size and morphology of the nuclei and structure of the cytoplasm. Furthermore, the number of inflammatory cells (e.g. neutrophils, lymphocytes, phagocytes, macrophages) is markedly reduced upon treatment with antioxidants according to the invention (FIG. 1 to 3).

In long term experiments by using mice intoxicated with DDC for 8-10 weeks the presence (or absence) of cell ballooning and/or Mallory bodies (MBs) in liver samples of treated animals is determined and compared with the control groups of animals (Example 3, FIGS. 4 to 6).

To determine the effect of these antioxidants in the treatment and/or prophylaxis of chronic liver metabolic diseases and epithelial cancers upon 10 weeks of intoxication with DDC or GF, respectively, test mice receive i.p. or i.v. (tail vein) injections comprising the antioxidant compounds according to the invention, e.g. Mito Q, MitoS or MitoVit E for subsequent 7 days, and compared with vehicle-injected control mice and other appropriate controls (see Example 3).

Alternatively, after 10 weeks of DDC intoxication, tested animals receive i.p. injections of MitoQ (1.25 mg/kg) twice within subsequent 7 days (day 1 and day 4 of the corresponding week), and are analysed by routine histology (standard haematoxylin/eosin staining according to Luna L. G., 1968, Manual of Histologic staining methods of the Armed Forces Institute of Pathology, 3rd edition. McGraw Hill, New York). The degree of cell ballooning and the number of Mallory bodies is greatly reduced in the MitoQ treated animals intoxicated with DDC when compared to appropriate controls (Example 3, see FIG. 4 to 6).

In further set of experiments, MitoQ, MitoS or MitoVit E is fed to mice intoxicated for 8 to 10 weeks with DDC or GF in their drinking water for subsequent 7 to 14 days (Example 3).

In some other experiments the antioxidant(s) according to the invention are applied to mice for 6 weeks of DDC- or GF intoxication followed by simultaneous treatment with MitoQ, MitoS or MitoVit E for subsequent 4 weeks by using 10 to 50% of maximum tolerated dosages of MitoQ, MitoS or MitoVit E respectively, and compared with control groups of animals intoxicated for 10 weeks solely with DDC or GF (see Example 3).

In another set of experiments, 10 week-intoxication of mice with DDC or GF is followed by 4 weeks of recovery. In this experiment it is further shown that the toxic memory effect (as a result of reexposure to DDC or GF intoxication for 24 to 72 hours) is reduced or abolished by simultaneous treatment with antioxidants according to the invention.

To evaluate the prophylactic effect of the antioxidants in liver disorders according to the invention, one group of DDC- or GF fed mice receives simultaneous treatment e.g. with 10 to 50% of the maximum tolerated dosages of MitoQ, MitoS or MitoVit E, respectively, and then is compared to a group of control animals being exposed for 10 weeks solely to DDC or GF (Example 3).

Alternatively, administration of e.g. MitoQ, MitoS or MitoVit E within an initial recovery period for 4 weeks is followed by subsequent 24 to 74 hours of intoxication with DDC or GF, wherein treated mice are compared to the control animals not treated by the antioxidants after 2.5 months of DDC- or GF exposure.

The application of the antioxidant(s), e.g. derivatives of coenzyme Q, vitamin E or a glutathione peroxidase mimetic, provides a significant reduction in morphologic abnormalities, e.g. hepatocyte ballooning, intracellular inclusions of misfolded proteins and MBs in liver(s) of DDC- or GF intoxicated animals. These results (Example 3, FIGS. 1 to 6) demonstrate that this cellular damage is mitigated by mitochondrial targeting of antioxidant compounds according to the invention. The DDC- or GF intoxicated mice models mimic observations made in the patients suffering from e.g. NASH or ASH and provide powerful in vivo and in vitro systems to study the role of antioxidants, e.g. derivatives of coenzyme Q₁₀ and vitamin E in the treatment or prophylaxis of diseases according to the invention.

Treatment and/or prophylaxis of human patients with liver disorders according to the invention with these mitochondrial targeted antioxidants significantly reduce liver pathology and thereby provide therapeutic and/or prophylactic efficacy as a treatment for these disorders.

In order to evaluate oxidative stress in control versus DDC-intoxicated mice with or without treatment by using antioxidants according to the invention, tocopherol quinone (TQ) content (Gille L. et al., 2004, Biochemic. Pharmacology, 68: 373-381) in isolated liver mitochondria (all tested animal groups prepared according to protocols in Example 3) is performed. The mouse liver mitochondria are prepared according to modified protocol from Staniek K. and Nohl H., 1999, Biochem. et Biophys. Acta, 1413: 70-80 ; Mela L. and Sietz S., 1979, Methods in Enzymology, Academic Press Inc.: 39-46, and TQ content is normalized to several parameters including protein and cytochrome concentrations and activity tests of complex I (NADH dehydrogenase), complex II (succinate dehydrogenase), complex III (cytochrome bcₗ) and complex IV (cytochrome oxidase). Overall, these experiments show the elevated TQ levels in DDC intoxicated mice when compared to controls and mice treated with MitoQ (Example 4).

In a further experimental set up to evaluate the oxidative stress induced proteins, western blot analysis of hemoxygenase (HO-1) expression level is employed by using extracts derived from DDC intoxicated mice treated simultaneously with MitoQ in short time exposure (3 days, Example 5). A marked reduction of DDC-induced overexpression of the HO-1 (known to be induced by reactive oxygen species (ROS), Suematsu M. and Ishimura Y., 2000. Hepatology, 31(1): 3-6) suggest that oxidative stress is greatly reduced in liver by antioxidants according to the invention (Example 5, FIG. 7).

Furthermore, the protein expression level of fatty acid binding protein (FABP) representing a sensitive marker for hepatocyte damage (Monbaliu D. et al., 2005, Transplant Proc., 37(1): 413-416) shows a significant decrease of FABP protein in DDC intoxicated mice when compared to the control group. Under MitoQ treatment of this group of animals the FABP protein expression is reaching almost control mice FABP expression values, thus suggesting again the effect of MitoQ in treatment or prophylaxis of diseases according to the invention (Example 5).

In another experimental set-up to investigate the effect of antioxidants according to invention in DDC- or GF intoxicated versus control mice, serum levels of liver specific enzymes are monitored as for example, in the Actitest (Biopredictive, Houilles, France) that provides a measure of liver damage and particularly fibrosis, which is characteristic of several diseases according to the invention (see Example 6). The serum levels of e.g. a₂-macroglobulin, haptoglobin, γ-glutamyl transpeptidase, total bilirubin, apolipoprotein A1 and alanine aminotransferase are measured from DDC- or GF treated, control, and corresponding DDC- or GF treated animals also exposed to the mitochondrially targeted antioxidants using the methods described in Poynard, et al., 2003, Hepatology 38:481-492, following general time line strategy according to Example 3.

Actitest performed also with human serum as a measure of liver damage, especially fibrosis, is similarly employed to monitor the effect of treatment of patients with these diseases with antioxidants according to the invention.

Alternatively, in serum from various tested animal groups following parameters indicating liver damage , namely bilirubin, alanine-aminotransferase (ALT/GPT), aspartate aminotransferase (ASAT/GOT) and glutamate dehydrogenase (GLDH) are determined according to standard protocols in clinical diagnostics employing commercially available kits (Example 6). The reduction of serum liver enzymes in animals (as e.g. alanine- and aspartate aminotransferases, see FIG. 8) treated with the compounds according to the invention indicates the reduction of liver damage in such treated samples and provides support for the therapeutic efficacy of these compounds in diseases according to the invention.

To evaluate the production of reactive oxygen species (ROS) one may, for example, employ dihydroethidium (DHE) staining of liver sections (e.g. frozen sections) prepared from control and DDC- or GF intoxicated animals according to a standard protocol (Brandes R P et al., Free Radic Biol Med. 2002; 32 (11): 1116-1122). This approach allows demonstration of induction of ROS production in vivo in livers of DDC- or GF intoxicated animals thus mimicking observations made in the patients suffering from the diseases according to the invention (Example 7). Other possibilities to evaluate the ROS formation in DDC- or GF fed mice include e.g. a lucigenin chemiluminescence assay (Goerlach A. et al., 2000, Circ Res., 87(1): 26-32).

This experimental set-up is further applied to DDC- or GF-fed animals treated with the targeted antioxidants according to the invention (Example 6). The general strategy of time-lines and dosage regime(s) for DDC- or GF intoxication of tested animals and for their treatment with the antioxidants is identical to the experimental approach used for determination of morphologic abnormalities, e.g. intracellular inclusions of misfolded proteins and MBs in livers of DDC- or GF intoxicated animals according to Example 3.

The application of the antioxidants, e.g. derivatives of vitamin E, coenzyme Q₁₀ or a glutathione peroxidase mimetic by using the general protocols according to Example 7 provides a significant reduction in ROS formation and thereof has a therapeutic benefit in liver disorders according to the invention (Example 8).

Optionally, in vitro experiments employing hepatoma cell lines (e.g. HepG2 or Hep3B), the SNU-398 cell line derived from a hepatocellular carcinoma (ATCC No. CRL-2233, LGC Promochem, Germany), the HUH-7 human carcinoma cells (Japanese collection of Research Biosources JCRB 0403) or the Tib-73 mouse embryonic cell line (American type collection, ATCC TIB 73=BNL CL2 derived from BAL/c mouse, MD, US) allows measurement of ROS production in liver cells upon DDC intoxication (Example 9). A glutathione synthesis inhibitor L-buthionine-(S,R)-sulfoximine (BSO) can be applied as an alternative to elevate endogenous oxidative stress (Kito M. et al., 2002, Biochem Biophys Res Commun., 291(4): 861-867).

Since CoCl₂ has recently been shown to affect mitochondria (Jung J Y and Kim W J., 2004, Neurosci Lett., 371:85-90) in order to measure ROS production in differentiated cell lines, HepG2 (ATCC No. HB-8065, MD, US) can be alternatively stimulated by 100 μM CoCl₂ (Sigma) (Bel Aiba R S, et al., 2004, Biol. Chem. 385: 249-57).

Another approach well established on cultured cells (as well as in isolated cell organelles or the entire tissue) allows measurement of the ROS production induced by Antimycin A (FIG. 10) according to Chem Biol Interact. 2000 Jul. 14; 127(3):201-217, or by rotenone using lucigenin chemiluminescence assay (Goerlach A. et al., 2000, Circ Res., 87(1): 26-32).

Liver cell cultures intoxicated for up to 3 days with DDC (conc=50 =g/ml of medium), BSO (up to 100 μM), or Antimycin A (or rotenone) or with CoCl₂ (100 μM) demonstrate induction of ROS production in vitro thus providing another suitable model mimicking observations made in patients suffering from the diseases according to the invention.

By employing standard protocols according to Example 9, the differentiated cell lines (e.g. hepatoma cells) intoxicated with DDC, BSO, Antimycin A (or rotenone) or CoCl₂ (100 μM) and simultaneously treated with MitoQ or MitoVit E, respectively (in concentrations corresponding to EC₅₀=0.51 nM for MitoQ and EC₅₀=416 nM for MitoVit E according to Jauslin M. L. et al., 2003, FASEB J., (13): 1972-4) or in latter case in concentrations ranging from 0.5 to 10 μM, provide a significant reduction in ROS formation, thus further confirming a therapeutic benefit of mitochondrially targeted antioxidants in liver disorders according to the invention (see FIG. 9, Example 10).

MBs are also found in chronic cholestatis such as primary biliary cirrhosis and primary sclerosing cholangitis. To determine the effect(s) of mitochondrially targeted antioxidants according to the invention in treatment and/or prevention of chronic cholestatic conditions the treatment paradigms described above for DDC- or GF intoxicated mice (Example 3) is followed. Recovered drug-primed animals are subjected to common bile duct ligation (CBDL) or feeding of a cholic acid (CA)-supplemented diet for up to 7 days (Fickert P. et al. 2002, Am. J. of Pathology, 161 (6): 2019-2026) with or without MitoQ and MitoVit E, respectively, and compared to appropriate control groups.

The general strategy to determine the effect(s) of mitochondrially targeted antioxidants in treatment and/or prevention of liver fibrosis and cirrhosis employs carbon tetrachloride (CCl₄)-induced liver damage in mouse or rat models (according to Arias I. M. et al., 1982. The Liver Biology and Pathobiology. Raven Press, New York) treated with antioxidants according to the invention.

To determine the effect(s) of mitochondrially targeted antioxidants according to the invention in treatment and/or prevention of epithelial cancers by following e.g. the treatment paradigms described above for DDC- or GF intoxicated mice but instead employs immunocompromised mice harbouring human epithelial cell cancer xenografts (nude mice tumor xenografts, as e.g. CD1 nu/nu mice from Charles Rivers Laboratories, USA). The tumors that are xenografted subcutaneously according to standard methods known in prior art (Li K. et al., 2003, Cancer Res., 63(13): 3593-3597) include but are not limited to colon adenocarcinomas, invasive ductal carcinomas of the breast small and non-small cell lung carcinoma, prostate tumors, pancreatic tumors and stomach tumors.

Treatment of such mice demonstrates reduced growth of tumors, increased necrosis of the tumors and decreased vascularization of the tumor xenografts. Similarly, the levels of ROS in nude mice tumor xenografts are monitored as described above and are reduced in xenograft tumors treated with the antioxidants according to the invention (Example 11).

When compared to the state of the art of therapy or prophylaxis of liver disorders and liver and other epithelial cancers the method of treatment according to the invention surprisingly provides an improved, sustained and more effective treatment.

The invention will be further illustrated below with the aid of the figures and examples, representing preferred embodiments and features of the invention without the invention being restricted hereto.

### BRIEF DESCRIPTION OF FIGURES

FIG. 1 to 3: Effect of MitoQ on the Degree of Hepatocyte Damage in Mouse Liver Upon Short Term (3 Days) Exposure to DDC

FIG. 1: Normal liver is characterized by hepatocytes mostly arranged in strands that are orientated to the central vein (annotated as a triangle) and sinusoids (C, original white colour with a few red dots representing erythrocytes) located between these strands of hepatocytes. The nuclei of the hepatocytes (A, in original blue in H&E stain) are large, not condensed and mostly show one prominent nucleolus, the cytoplasm (annotated as B, is stained relatively homogeneously pink, H&E staining). No infiltration with lymphocytes or granulocytes around portal vein (annotated as asterisk) is detected (magnification 200×).

FIG. 2: After intoxication with DDC for 3 days the architecture of the liver is severely damaged: the orderly arrangement of the hepatocytes is lost. Especially around the portal vein (annotated by asterisk) infiltrates with lymphocytes and granulocytes are seen (annotated by arrow). The hepatocytes show different indications of cell damage: the cells loose their contact to other cells, the nuclei are condensed and the cytoplasm gets bluish-pink as indication for apoptosis. The cells increase in size (ballooning) and the cytoplasm becomes inhomogeneous, clumps of cytokeratin are visible. In addition, the cells loose their plasma membrane as another indication for necrosis. The annotation A, B, C is identical to FIG. 1. Around the portal vein (marked by asterisk) inflammatory cells (marked by arrow) and damaged hepatocytes (no clear cell boundaries discernible, cell swelling) are detected. Deposits of protoporphyrin (small brown dots) represent a DDC-specific effect on protohaem ferrolyase. The annotation A, B, C is identical to FIG. 1 (magnification: 400×).

FIG. 3: After simultaneous treatment with MitoQ (MitoQ in PBS/1% DMSO (225 mmol/animal/day corresponding to 6 mg/kg) the normal architecture again is visible with strands of hepatocytes bordered by sinusoids. The morphology of the hepatocytes is normal regarding size and morphology of the nuclei and structure of the cytoplasm (Example 3). Absence of inflammatory cells around the portal vein (marked by asterisk); except of slight indication for cell swelling and deposition of protoporphyrin hepatocytes look normal. The annotation A, B, C is identical to FIGS. 1 and 2 (magnification: 400×).

FIG. 4 to 6: Effect of MitoQ on the Degree of Hepatocyte Damage in Mouse Liver Upon Long Term (10 Weeks) Exposure to DDC

FIG. 4: In normal non DDC-intoxicated mice (4 month of age) liver structure in general resembles that of young non DDC-intoxicated mice depicted in FIG. 1A (see A=nuclei, B=cytoplasm, C=sinusoids). Inflammation around the portal vein (asterisk) is absent and hepatocytes are arranged in strands. The cytoplasm of the cells is regularly stained and of even size; no ballooning of the cells or Mallory body formation is seen (magnification: 400×).

FIG. 5: Structure are identically as in FIGS. 1 to 4 (see A=nuclei (original blue), B=cytoplasm (original pink), C=sinusoids (original white with red dots)) and D original brown colour represents pigment (predominantly protoporphyrin) in the bile ducts. After intoxication with DDC for 10 weeks and subsequent recovery without DDC for one week the arrangement of the hepatocytes is still disturbed. The hepatocytes show various degrees of cellular damage ranging from disintegration of the cytoskeleton to cell ballooning and formation of Mallory bodies (arrow). Accumulation of protoporphyrin in seen especially in bile ducts (arrowhead), magnification: 400×.

FIG. 6: After 10 weeks DDC intoxication in the recovery period (no DDC for 1 week) treatment with two injections of MitoQ is performed. The improvement during the recovery is significant; only slight alterations of hepatocyte morphology are seen. The majority of the hepatocytes looks normal and cell ballooning and/or Mallory body formation is absent. The accumulation of protoporphyrin in bile ducts in the vicinity of portal vein (asterisk) is also markedly reduced. Structures are identically as in FIGS. 1 to 5 (see A=nuclei (original blue), B=cytoplasm (original pink), C=sinusoids (original white with red dots) and D original brown represents pigment (predominantly protoporphyrin)). Magnification: 400×.

FIG. 7: Expression of the Inducible Form of Hemoxygenase (HO-1) in DDC Intoxicated Mice Treated with MitoQ

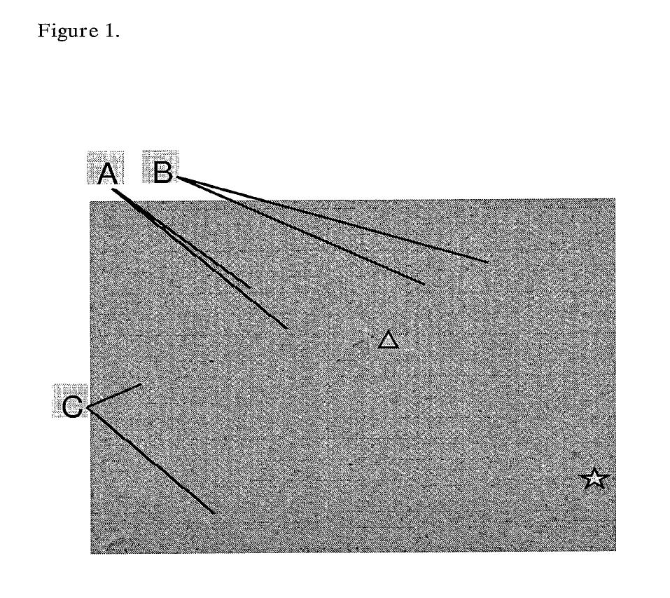

Western blot analysis of HO-1 (32 kDa, annotated by arrow) shows a marked induction under DDC intoxication (lanes no. 4, 5, 6 representing solvent controls with DDC), whereas treatment with MitoQ (lanes 7, 8=112 nmol/kg MitoQ without DDC and lanes 9, 10=112 nmol/kg MitoQ with DDC) result in strong reduction of HO-1 protein expression. Lanes No. 1 to 3 represents solvent controls without DDC. The low molecular weight protein marker (22, 36, 55, 64, 98 and 148 kDa) is used.

In order to normalize HO-1 protein expression in lanes 1 to 10, comparison to the constitutively expressed isoform HO-2 (36 kDa) by using Chemiimager 5500 software (Alpha Innotech) is performed showing 7 fold reduction of HO-1 in DDC intoxicated animals treated with MitoQ when compared to the control group represented by DDC intoxicated mice. Overall, in this set of experiments DDC intoxicated mice (for 3 days) daily injected (i.p.) with MitoQ in PBS/1% DMSO are analysed and compared to appropriate controls.

FIG. 8: Serum Parameters of DDC Intoxicated Mice Under Simultaneous MitoQ Treatment

In serum from various animal groups the activity of serum liver enzymes indicating liver damage, namely bilirubin, alanine aminotransferase (ALT/GPT; in diagram represented by white bars), aspartate aminotransferase (ASAT/GOT in diagram represented by black bars) are determined according to standard protocols in clinical diagnostics by employing commercially available kits (No: 11552414; 11876805216; 11876848216 all purchased by Roche AG, Switzerland) on a Hitachi/Roche 917 Analyser.

Lanes: no. 1 and 2 represent non DDC intoxicated group of animals and DDC intoxicated mice, respectively. Lanes 3 to 5 represent DDC intoxicated (3 days) and simultaneously MitoQ treated animals with concentrations of 3-, 6- and 12 mg/kg. The most prominent reduction of enzymatic activity shows alanine aminotransferase (ALT/GPT annotated by white bar), followed by aspartate aminotransferase (AST/GOT annotated by black bar) whereas bilirubin activity remains without any changes (data not shown).

FIG. 9: ROS Production by 100 μM CoCl2 (0, 10, 20, 30 Minutes) in HepG2 Cells Simultaneously Treated with MitoQ

5 μM MitoQ is able to reduce basal ROS production already in unstimulated cells. (see lane 2). CoCl₂-induced ROS production (100 μM CoCl₂) is decreased by 5 μM MitoQ. These results demonstrate that 5 μM MitoQ can significantly decrease basal and CoCl₂-stimulated ROS levels in HepG2 cells (Example 10). The annotation “A” (lanes 4, 5, 6) stands for HepG2 cells stimulated with CoCl₂. X axis represents a concentration range of MitoQ [μM] and y axis the relative DCF Fluorescence [%]. *p<0.05 vs unstimulated (0 μM MitoQ); # p<0.05 vs CoCl₂.

FIG. 10: Stimulation of HUH-1 Cell with 1 μM Antimycin Using Lucigenin Chemiluminescence Assay

HUH-7 cells are incubated in 6 well plates and stimulated with Antimycin A in concentration 0-25 μM (0, 1 and 5 μM) simultaneously with or without MitoQ in concentration range from 0 to 1000 nmol dissolved in DMEM (Gibco) for 3 hours at 37° C. The light reaction between superoxide and lucigenin is detected. X axis represents a concentration range of MitoQ [nM] whereas y axis the chemiluminescence signal expressed as average counts per minute [cpm] after normalization to cell number determined by cell counter. Overall, this diagram shows a significant reduction in ROS formation, thus further confirming a therapeutic benefit of mitochondrially targeted antioxidants in liver disorders according to the invention.

### EXAMPLES

### Example 1

### Experimental Induction of Mallory Bodies (MBs)

MBs can be induced in mouse livers by chronic intoxication of various mouse strains: e.g., Male Swiss Albino mice: strain Him OF1 SPF (Institute of Laboratory Animal Research, University of Vienna, Himberg, Austria) with 3,5-diethoxycarbonyl-1,4-dihydrocollidine (1,4-dihydro-2,4,6-trimethylpyridine-3,5-dicarbonic acid diethyl ester, DDC, Cat. no. 13703-0, Sigma-Aldrich Steinheim, Germany) or Griseofulvin (GF, Cat. no. 85,644-4, Sigma-Aldrich).

The standard diet (Sniff Spezialdiäten GmbH, Soest, Germany) containing 2.5% GF or 0.1% DDC is produced as pellets by Sniff.

Animals are kept in conventional cages or in sterile isolators with a 12 hrs day-night cycle. Animals receive humane care according to the criteria outlined in the “Guide for the Care and Use of Laboratory Animals” prepared by the National Academy of Sciences and published by the National Institutes of Health; NIH publication 86-23, revised 1985.

Mice (8 weeks old) are fed a standard diet containing either 0.1% DDC or 2.5% GF for up to 2.5 months.

Mouse livers respond to DDC- or GF intoxication first with ballooning of hepatocytes and formation of a denser keratin IF network. After around 6 weeks of intoxication, ballooned hepatocytes show a reduced density of the keratin IF and early MBs can be observed as fine granules associated with the keratin IF network. Continuation of intoxication leads to the appearance of large MBs typically located in the perinuclear cytoplasmic region. Most hepatocytes containing large MBs have a markedly reduced or even undetectable cytoplasmic IF keratin network. Upon cessation of intoxication, MBs disappear within several weeks. At 4 weeks of recovery from intoxication, there are groups of hepatocytes devoid of cytoplasmic keratin filaments but still containing small remnants of MBs at the cell periphery in association with desmosomes. If such mice are reexposed to DDC or GF numerous MBs reappear within 24 to 72 hours (Stumptner C. et al., 2001, J. Hepatol., 34: 665-675). This enhanced formation of MBs upon reintoxication was interpreted—in analogy to allergic reactions—as a toxic memory effect.

Mice are killed at different time-points of intoxication by cervical dislocation and the livers are either immediately snap-frozen in methylbutane precooled with liquid nitrogen for immunofluorescence or fixed in 4% buffered formaldehyde for routine histology and immunohistochemistry.

### Example 2

### Evaluation of Liver Alterations; Detection of Mallory Bodies (MBs)

Liver samples prepared according to Example 1 are used for simple histologic staining such as with haematoxylin and eosin (Luna L. G., 1968, Manual of Histologic staining methods of the Armed Forces Institute of Pathology, 3rd edition. McGraw Hill, New York). Furthermore, single-label immunohistochemistry or double-label immunoflourescence microscopy is performed to detect MBs in tested animals.

A) Single-label immunohistochemistry on paraffin-embedded sections: Sections (4 μm thick) are deparaffinized in xylene and rehydrated in graded ethanol (100%, 90%, 80%, 70%, 50% ethanol) and PBS (50 mM potassium phosphate, 150 mM NaCl, pH 8.0-8.5). For antigen retrieval, rehydrated sections are incubated with 0.1% protease type XXIV (Sigma Steinhein, Germany) for 10 min at room temperature (for ubiquitin Dako primary antibodies), or microwave (conventional household microwave oven with energy control) at 750 W for 10 min in 10 mM citrate buffer, pH 6.0 (for the polyclonal K8/18 antibody 50K160, the monoclonal K8 antibody K8.8 [Neomarkers], the monoclonal K18 antibody DC-10 [Neomarkers] and p62CT: polyclonal guinea pig antibody against C-terminal peptide sequence of p62; Zatloukal K. et al., 2002, Am. J. Pathol., 160: 255-263). After washing in PBS, endogenous peroxidase is blocked by incubation in 1% H₂O₂ (Merck) in methanol for 10 min and washed subsequently in PBS. In the next step sections are incubated with primary antibodies in a humidified chamber (Nunc) for 60 min at room temperature and washed three-times with PBS. Then the sections are incubated with Multi Link Swine anti-Goat, Mouse, Rabbit immunoglobulins (Dako) diluted 1:100 in PBS for 30 min at room temperature, washed three-times with PBS and incubated with Streptavidin biotin horse radish peroxidase complex ABC/HRP (Dako; Sol A 1:100 and Sol B 1:100 in PBS) for 30 min. Alternatively, incubation with peroxidase-conjugated rabbit anti guinea pig immunoglobulins secondary antibody (Dako) diluted 1:100 in PBS for 30 min is performed followed by three-times washing with PBS. Subsequently tyramide amplification is performed by applying biotinyl tyramide solution 1:50 in amplification diluent (TSA™ Biotin System, NEN, Boston, Mass., USA) for 5 min, washed three-times with PBS and followed by incubation with streptavidin-peroxidase solution (1:100 in PBS) for 30 min.

P62CT antibody binding is detected using the TSA™ Biotin System. Reactivities of ubiquitin and K8/18 antibodies are detected using the ABComplex system (Dako), rinsed in tap water followed by application to the section of a cover slip with the mounting medium Aquatex® (Merck).

For colour development, incubation with 3-amino-9-ethylcarbazole (AEC, Dako) for 5 min is to be performed, followed by three-time wash in PBS and counterstaining with Mayr's haemalaun with subsequent rinsing with tap water and mounting of a cover slip with Aquate® (Merck).

B) double-label immunofluorescence microscopy on frozen section: Cryosections (3 μm thick) are cut using Cryocut (Leica CM3050, Leica, Nuβloch, Germany), air-dried and fixed in acetone at −20° C. for 10 min. Alternatively (particularly if preservation of nuclear architecture is required) sections are fixed in PBS-buffered 4% formaldehyde for 15 min at room temperature, followed by acetone fixation for 5 min at −20° C. Sections are air-dried after fixation or rinsed in PBS.

Subsequently, first primary antibody p62CT (polyclonal guinea pig antibody against C-terminal peptide sequence of p62 (Zatloukal K. et al., Am. J. Pathol., 2002, 160: 255-263), antibodies to K8 (Ks 8.7, Progen, Heidelberg, Germany), K18 (Ks 18.04, Progen), K8/18 (50K160), and ubiquitin (ID Labs Inc., London, ON, Canada) is applied for 30 min at room temperature in a wet chamber (Bioassay plate, Nune, Roshilde, Denkmark). Alternatively, the antibodies are applied over night at 4° C., followed by three-time wash with PBS for 5 mm.

In the next step a first secondary antibody is applied for 30 min at room temperature in a humidified chamber under light protection followed by three-times wash with PBS for 5 min. Application of a second primary antibody for 30 min at room temperature in a wet chamber under light protection is followed again by three-times washing with PBS for 5 min. Further application of a second secondary antibody for 30 min at room temperature is performed in a wet chamber under light protection followed again by three-times washing with PBS for 5 min. After the last antibody incubation, slides are rinsed with distilled water and then with ethanol for a few seconds and air-dried.

Secondary antibodies to be used are, e.g., fluorescein isothiocyanate (FITC)-conjugated goat anti-mouse IgG (Zymed, San Francisco, Calif., USA) or Alexa 488 nm-conjugated goat anti-mouse IgG (Molecular Probes, Leiden, The Netherlands) and tetramethylrhodamine isothiocyanate (TRITC)—or FITC-conjugated swine anti-rabbit Ig (Dako, Glostrup, Denmark) and TRITC-conjugated rabbit anti guinea pig Ig (Dako)

Finally, specimens are mounted with Mowiol (17% Mowiol 4-88 [Calbiochem Nr. 475904], 34% glycerol in PBS) or other commercially available mounting medium.

All antibodies are diluted in PBS and applied separately in sequential incubations. Fluorochrome-conjugated antibodies are centrifuged at 16,000×g for 5 min to remove aggregates before application onto slides. For negative control, first antibodies are replaced by PBS, pre-immune serum or isotype-matched immunoglobulins, respectively.

Immunofluorescent specimens are analyzed with a laser scanning microscope (LSM510 laser-scanning microscope, Zeiss, Oberkochen, Germany). For colocalization analyses (dual labeling) images are acquired using the multitrack modus. Merged pictures appear in green/red pseudo-colour with yellow colour at sites of co-localization. Slides are stored protected from light at +4° C.

### Example 3

### Effect of the Antioxidants According to the Invention on Liver Pathology

To evaluate the impact of the antioxidants according to the invention on regression of morphological alterations in early stages of DDC- or GF intoxicated mice livers a positive control group of animals (3 to 7 days exposure to DDC or GF only) is compared with DDC- or GF intoxicated mice treated for further 3 to 7 days with MitoQ a mixture of MitoQuino1 [10-(3,6-dihydroxy-4,5-dimethoxy-2 methylphenyl)decyl]triphenylphosphonium bromide and MitoQuinone [10-(4,5-dimethoxy-2-methyl-3,6-dioxo-1,4-cyclohexadien-1-yl)decyl]-triphenylphosphonium bromide (provided by Key Organics Ltd, London, UK), or MitoVit E [2-(3,4-dihydro-6-hydroxy-2,5,7,8-tetramethyl-2H-1-benzopyran-2-yl)ethyl]-triphenylphosphonium bromide (provided by Key Organics Ltd, London, UK), respectively.

For injections, MitoQ or MitoVit E is dissolved in PBS supplemented with sufficient DMSO preferably 1%) to maintain solubility of antioxidants. Intraperitoneal or i.v. (tail vein) injections are given to pairs of mice and compared with vehicle-injected controls. These correspond to maximum tolerated dose of 20 mg of MitoQ/kg/day (750 nmol) and 6 mg of MitoVit E/kg/day (300 nmol) according to Smith R. A. J et al., 2003, PNAS, 100 (9): 5407-5412.

MitoQ or MitoQ derivatives such as MitoS (a mixture of MitoQuino1 [10-(3,6-dihydroxy-4,5-dimethoxy-2 methylphenyl)decyl]triphenylphosphonium methane sulfonate and MitoQuinone [10-(4,5-dimethoxy-2-methyl-3,6-dioxo-1,4-cyclohexadien-1-yl)decyl]-triphenylphosphonium methane sulfonate or MitoVit E is supplemented to the diet. Doses are determined by measuring water or liquid diet consumption and mouse weight. Mice are fed in their drinking water for 3 to 7 days without any gross signs of toxicity with 500 μM or 1 mM MitoQ or MitoS (maximum tolerated doses of 232 μmol/kg/day or 346 μmol/kg/day respectively, corresponding to 154 and 230 mg/kg/day for the 500 μM and 1 mM diets), or with 500 μM MitoVit E (a maximum tolerated dose of 105 μmol/kg/day corresponding to 60 mg of MitoVit E/kg/day) according to Smith R. A. J. et al., 2003, PNAS, 100 (9): 5407-5412.

In a further test a group of DDC- or GF intoxicated animals are simultaneously treated with MitoQ (MitoS) or MitoVit E for 3 to 7 days and compared to control group exposed for 3 to 7 days to DDC or GF only.

In another set of experiments 3 to 12 mg/kg of MitoQ dissolved in 1% of DMSO in PBS is given intraperitoneally simultaneously to DDC intoxicated mice for 3 days and compared to control animals (positive control group represents DDC intoxicated mice whereas negative control represents non DDC intoxicated but vehicle injected animals).

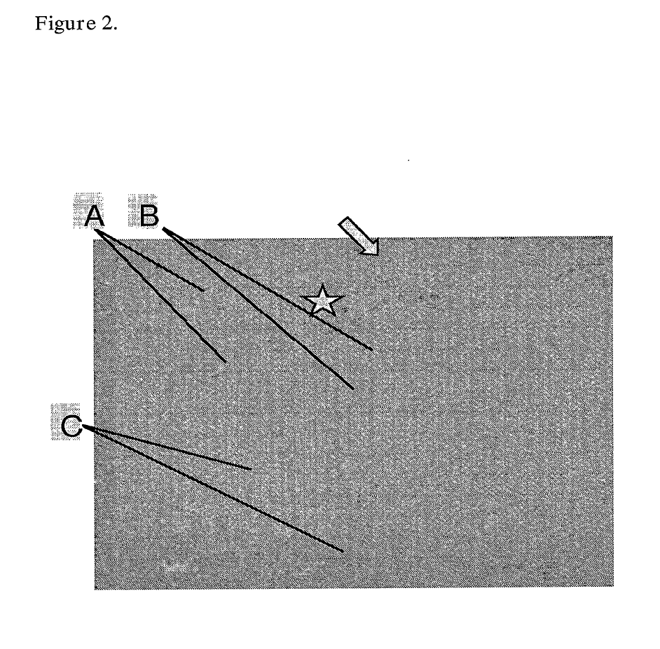

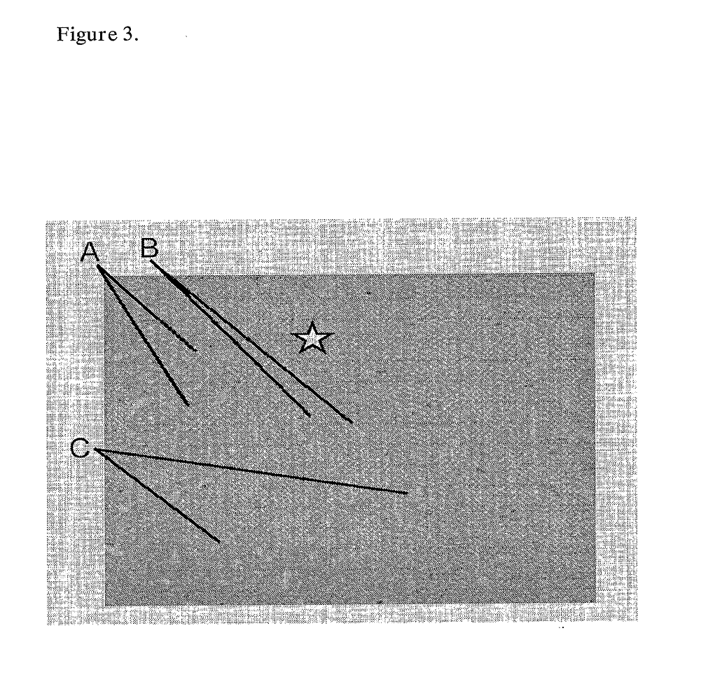

To practically assess in these short-term experiments the impact of mitochondrially targeted antioxidants according to the invention (e.g. MitoQ or MitoS) the presence (or absence) of inflammatory cells around the portal vein (Glisson's trias) and the degree of hepatocyte damage such as necrosis, collapse of cytoskeleton (see FIGS. 2 and 3) instead of cell ballooning and/or Mallory bodies analysis (typical for long term exposure to DDC or GF, respectively) are compared to the positive control. Both cell ballooning and Mallory bodies are not suited in these experiments due to the fact that they are not formed within this short time exposure to DDC to a degree that allows statistical evaluations.

Overall, under MitoQ treatment the normal architecture represented by strands of hepatocytes bordered by sinusoids is again visible. The morphology of the hepatocytes is normal regarding size and morphology of the nuclei and structure of the cytoplasm. Furthermore, the number of inflammatory cells (e.g. neutrophils, lymphocytes) is markedly reduced upon treatment with antioxidants according to the invention.

In long term experiments by using mice intoxicated with DDC (for 8-10 weeks) the presence (or absence) of cell ballooning and/or Mallory bodies (MBs) in liver samples of treated animals is determined and compared to the control groups of animals.

In one set of experiments, upon 10 weeks of intoxication with DDC or GF, tested animals receive i.p. or i.v. (tail vein) injections of MitoVit E or MitoQ (or MitoS) for subsequent 7 days given to pairs of mice and compared with vehicle-injected controls.

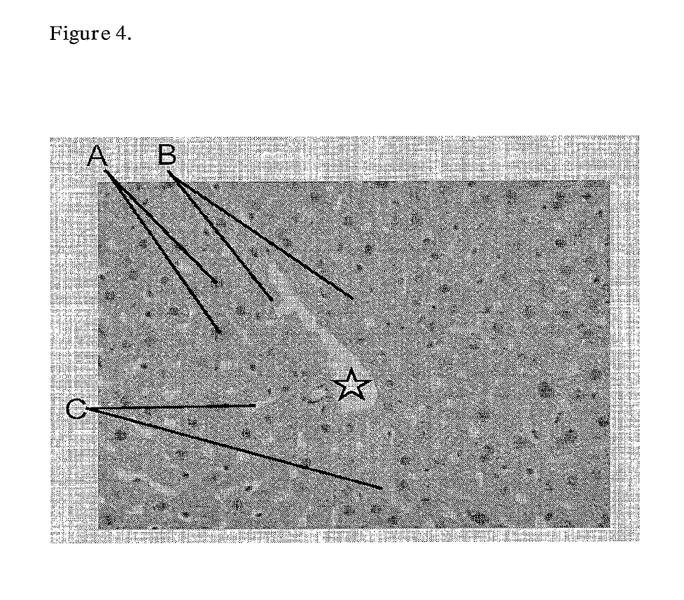

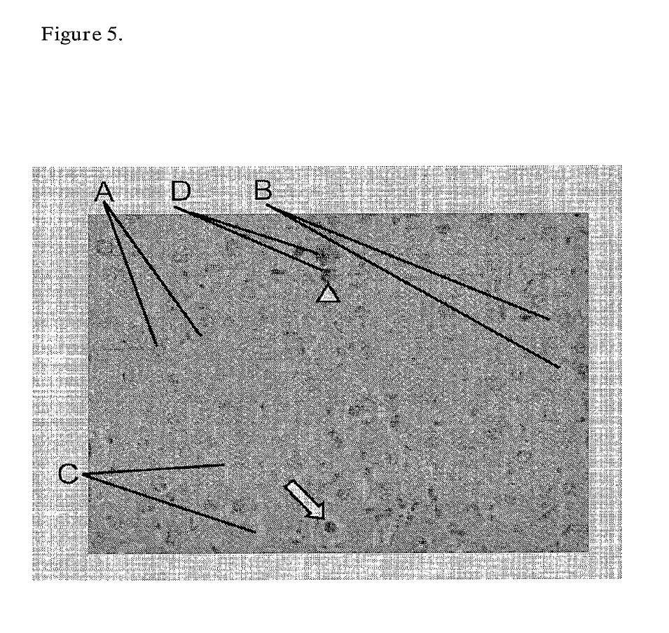

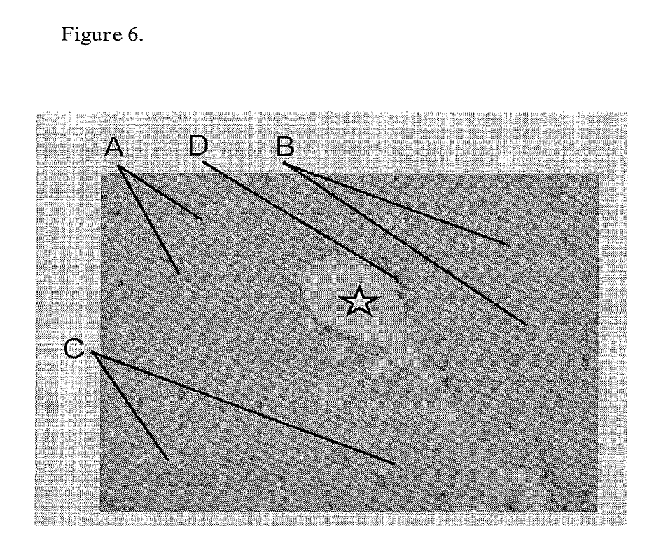

In another set of experiments, after 10 weeks of DDC intoxication tested animals receive i.p. injections of MitoQ (1.25 mg/kg) twice within subsequent 7 days (day 1 and day 4 of the corresponding week), and are analysed by routine histology (standard haematoxylin/eosin staining according to Luna L. G., 1968, Manual of Histologic staining methods of the Armed Forces Institute of Pathology, 3rd edition. McGraw Hill, New York). The degree of cell ballooning and the number of Mallory bodies is greatly reduced in the MitoQ treated animals intoxicated with DDC when compared to appropriate controls (FIG. 4 to 6).

In further set of experiments, MitoQ (MitoS) or MitoVit E is fed to mice intoxicated for 8-10 weeks with DDC or GF in their drinking water for subsequent 7 to 14 days.

In yet another set of experiments MitoQ (MitoS) or MitoVit E is applied to intoxicated mice for 6 weeks with DDC or GF simultaneously with further DDC or GF for subsequent 4 weeks by using 10 to 50% of maximum tolerated dosages of MitoVit E or MitoQ (MitoS), respectively, and compared with control groups of animal intoxicated for 10 weeks solely with DDC or GF.

In another set of experiments, 10 week-intoxication of mice with DDC or GF is followed by 4 weeks of recovery. Subsequent simultaneous reintoxication with DDC or GF and treatment with MitoVit E or MitoQ/MitoS reveals that the toxic memory effect (as a result of reexposure to DDC or GF intoxication for 24 to 72 hours) is reduced or abolished by treatment with the mitochondrially targeted antioxidants.

To evaluate the prophylactic effect of the antioxidants in liver disorders one group of DDC- or GF fed mice receives simultaneous treatment with 10 to 50% of the maximum tolerated dosages of MitoQ, MitoS or MitoVit E, respectively, and is then compared to the control animals being exposed solely to 10 weeks of DDC- or GF intoxication.

In one set of experiments administration of MitoQ, MitoS or MitoVit E within the initial recovery period (4 weeks) is followed by subsequent 24 to 72 hours of intoxication with DDC or GF, and compared to control non-treated animals.

It will be apparent to those skilled in the art that various modifications of the general protocols can be made.

Overall, in both short and long term intoxication with DDC or GF, respectively, the pronounced alterations of the liver are greatly ameliorated or reduced by the application of the antioxidants according to the invention used for the treatment or prophylaxis of liver diseases and/or epithelial cancers.

### Example 4

### Measurement of Oxidative Stress (Determination of Tocopherol Quinone Content) in Isolated Mitochondria Derived from DDC Intoxicated Mice with or without Treatment by Antioxidants According to the Invention

4.1. Isolation of Mouse Liver Mitochondria (MLM)

A method for rat heart mitochondria (Staniek K. and Nohl H., 1999, Biochem. et Biophys. Acta, 1413: 70-80 ; Mela L. and Sietz S., 1979, Methods in Enzymology, Academic Press Inc.: 39-46) is adapted for mouse liver (ca. 10% weight compared to rat liver) isolated from various animal groups according to Example 3. The isolation of liver is performed at 4° C. Each liver is cut into pieces and shock-frozen in liquid nitrogen (N₂) for storage. After thawing in preparatory buffer (0.3 M sucrose, 1 mM EDTA, 20 mM triethanolamine pH 7.4) plus 10 mg/L BHT (di-tert.butyl-hydroxytoluene) and 1 mM diethylenetriaminepentaacetic acid (Fe chelator) to prevent tocopherol oxidation, the tissue is cut into small pieces, 4× washed with prep. buffer, 5× gently homogenized in 15 ml buffer with a Potter pistil, diluted to 30 ml and centrifuged at 570 g for 10 min. The supernatant is filtered through 2 layers of cheesecloth. The mitochondria are pelleted at 7400 g for 10 min, gently resuspended by hand in 30 ml buffer, repelleted and washed again as above, finally resuspended in approximately 200 ml buffer. The protein concentration is measured with the Biuret method (BSA as standard, at least 200 mg protein needed for double determination) with expected yield of 3 to 6 mg.

For normalization purposes, the cytochrome concentration is calculated from the dithionite-reduced minus air-oxidized difference spectrum after solubilization of the membranes with 0.2% (v/v) Triton X-100 (Aminco DW2000 photometer, ca. 0.5-1 mg mitochondrial protein needed for double determination) (Williams J. N., Jr., 1964, Archives of Biochemistry and Biophysics, 107: 537-543); expected concentration of Cyt (a+a₃), Cyt c, Cyt c₁ and Cyt b in healthy mitochondria: 0.1-0.3 nmol/mg prot. each, extrapolated from rat liver mitochondria (Wakabayashi T. et al., 2000, Pathology International, 50:20-33).

As a control, an aliquot of the raw homogenate (after the first homogenization) containing all cellular membranes should be kept. The total membranes including the lightest fraction (microsomes) are pelleted at 165 000 g for 40 min (ultracentrifuge) according to Murias M. et al., 2005, Biochemical Pharmacology, 69: 903-912, washed and repelleted in 5 mL prep. buffer, and finally resuspended in ca. 200 mL buffer. The protein concentration is measured as above.

4.2. Analysis of Tocopherol Quionone (TQ)

Whole MLM (see paragraph 4.1.) can be used. The amount of 2-5 mg protein (mitochondria, total membranes or various fractions) in 1 ml H₂O is mixed with 5 mM SDS and 2 nmol UQ₆ (ubiquinone-6, as internal standard) and extracted with 3 ml anaerobic ethanol/hexane (2:5). The organic phase is evaporated under argon and the residue is dissolved in 120 ml ethanol. 40 ml is used for HPLC analysis (double analysis per sample) on a Waters LC1 module with a C18 column. Quinones and tocopherol are eluted with 50 mM NaClO₄ in ethanol/methanol/acetonitrile/HClO₄ (400:300:300:1) at 1 ml/min and detected optically (268 nm for TQ, 275 nm for UQ₆ and endogenous UQ₉ and UQ₁₀) or electrochemically (+0.6 V, for tocopherol and quinols) according to Gille L. et al., 2004, Biochemical Pharmacology, 68: 373-381; expected TQ content in healthy mitochondria is 1-5% relative to tocopherol or ubiquinone (Gille L. et al., 2004, Biochemical Pharmacology, 68: 373-381).

Overall, these experiments show the elevated TQ levels in mitochondria of DDC intoxicated mice when compared to controls and DDC mice treated with antioxidants according to the invention

4.3. Analysis of Additional Enzyme Complexes in Mouse Liver Mitochondria (MLM)

MLM derived from various groups of animals (see protocols in Example 3) are frozen and thawed 2-3 times to break the membranes and give access to various reagents (see below) according to Fato R. et al., 1996, Biochemistry, 35: 2705-2716). The photometric assays can be performed at 25° C. (Aminco DW2000 dual-wavelength photometer), ca. 5-20 mg mitochondrial protein are needed per assay:

a) Aconitase (marker for superoxide damage) (James A. M. et al., 2005, JBC, published on Mar. 23, 2005 as Manuscript M501527200). The assay contains 0.6 mM MnCl₂, 5 mM Na citrate, 0.2 mM NADP⁺, 0.1% Triton X-100 0.4 U/mL isocitrate dehydrogenase and 50 mM Tris pH 7.4. NADPH generation is followed at 340 to 410 nm; expected activity in healthy mitochondria: ca. 60 nmol/min per mg of isolated mitochondria according to Senft A. P. et al., 2002, Toxicology and Applied Pharmacology 178: 15-21.

b) Complex I (NADH dehydrogenase) (modified from Estomell E. et al., 1993, FEBS, 332, No. 1, 2: 127-131): The assay contains 0.1 mM NADH, 0.05 mM decylubiquinone, 2 mM KCN, 20 mM antimycin A and 20 mM Tris pH 7.5. The NADH decay is followed at 340 to 410 nm. Inhibition by 2 mg/mL rotenone corrects for unspecific quinone reduction; expected activity in healthy mitochondria: ca. 100-300 nmol/(min·mg) according to Stuart J. A. et al., 2005, Free Radical Biology & Medicine, 38: 737-745 and Barreto M. C., 2003, Toxicology Letters, 146: 37-47.

c) Complex II (succinate dehydrogenase) (modified from Gille 2001): The assay contains 2 mM succinate, 0.05 mM decylubiquinone, 2 mM KCN, 20 mM antimycin A and 20 mM Tris pH 7.5. The quinone decay is followed at 275 minus 320 nm. Inhibition by 25 mM malonate corrects for unspecific quinone reduction; expected activity in healthy mitochondria: ca. 70-100 nmol/min per mg of isolated mitochondria according to Barreto M. C., 2003, Toxicology Letters, 146: 37-47.

d) Complex III (cytochrome bc₁) (modified protocol according to Stuart J. A. et al., 2005, Free Radical Biology & Medicine, 38: 737-745). The assay contains 0.05 mM decylubiquinol (prepared from decylubiquinone by dithionite reduction and hexane extraction), 15 mM Cyt c, mM KCN and 20 mM Tris pH 7.5. Cyt c reduction is followed at 550 minus 540 nm. Inhibition by 20 mM antimycin A corrects for unspecific quinol oxidation; activity in healthy mitochondria: ca. 80 nmol/min per mg of isolated mitochondria according to Stuart J. A. et al., 2005, Free Radical Biology & Medicine, 38: 737-745).

e) Complex IV (cytochrome oxidase) (modified from Stuart J. A. et al., 2005, Free Radical Biology & Medicine, 38: 737-745). The assay contains 15 mM reduced Cyt c (prepared by dithionite reduction and air oxidation of excess dithionite) and 20 mM Tris pH 7.5. Cyt c oxidation is followed at 550 minus 540 nm; activity in healthy mitochondria: ca. 1 mmol/min per mg of isolated mitochondria (Stuart J. A. et al., 2005, Free Radical Biology & Medicine, 38: 737-745).).

### Example 5

### Evaluation of Oxidative Stress Induced Proteins (Hemoxygenase 1)

To evaluate hemoxygenase 1 (HO-1) protein expression know to be induced by oxidative stress (Suematsu M. and Ishimura Y., 2000, Hepatology, 31(1): 3-6) standard western blot analysis is performed using protein extracts derived from DDC intoxicated mice treated simultaneously for 3 days with MitoQ (diluted in 1% DMSO in PBS) or just vehicle itself (see protocols in Example 3).

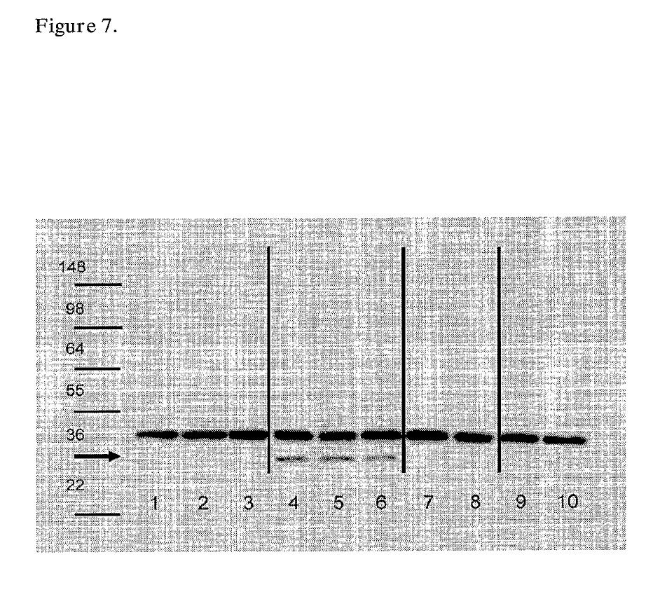

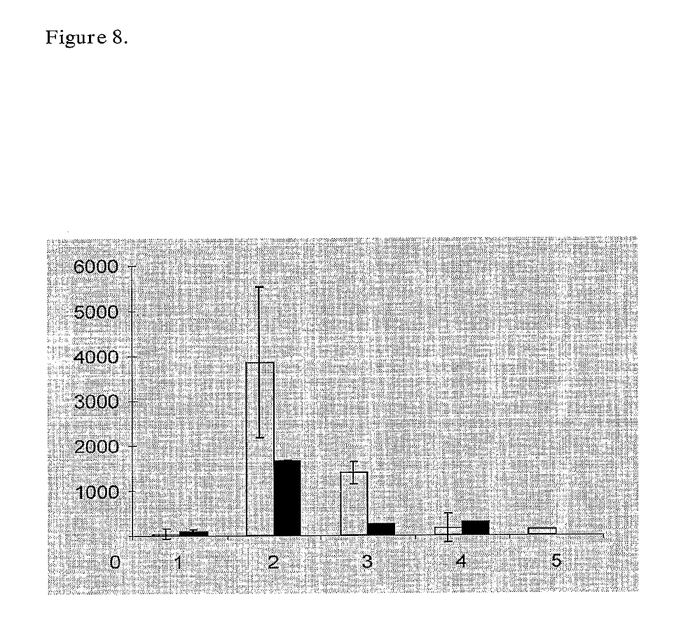

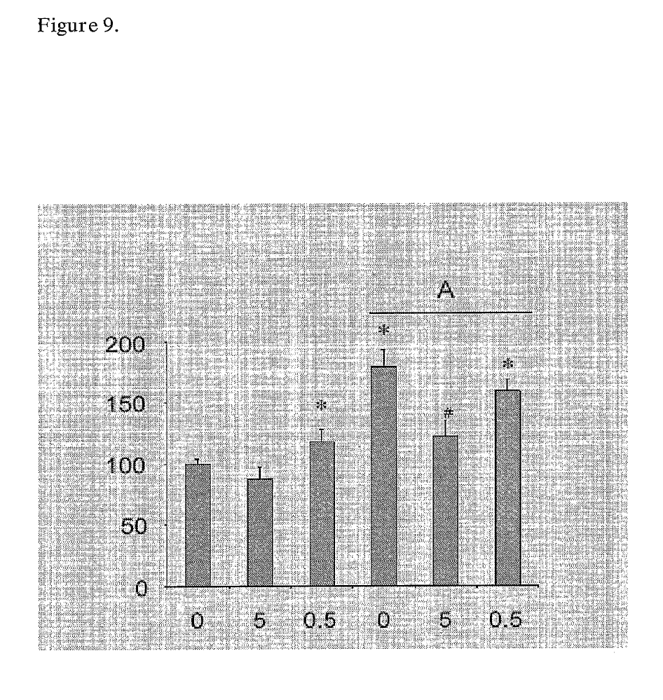

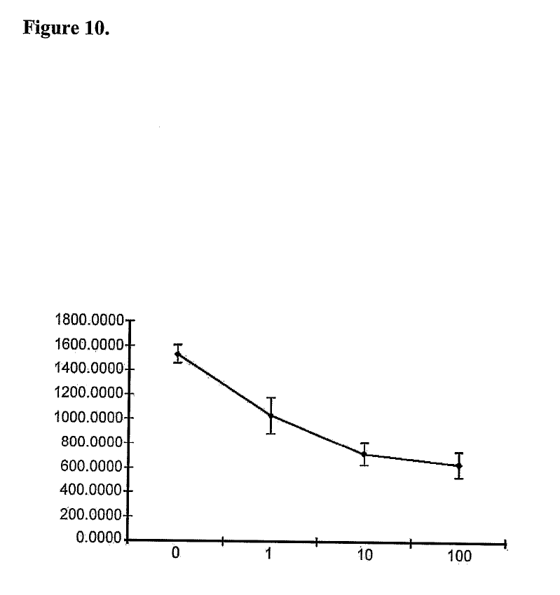

Liver tissues are resuspended in ice-cold RIPA-buffer (50 mM Tris-HCl pH 7.4, 250 mM NaCl, 0.1% SDS, 1% deoxycholate, 1% NP-40) supplemented with 2 μg/ml leupeptin, 2 μg/ml pepstatin, 2 μg/ml aprotinin, 1 mM phenylmethylsulfonylfluoride (PMSF), and 2 mM dithiothreitol followed by homogenization through sonication (2 bursts of 5 seconds) on ice. After incubation for 20 minutes on ice, the lysates are cleared by two centrifugational steps in a microcentrifuge at 13 000 rpm for 15 minutes at 4° C. and the supernatants are collected. Protein concentrations are determined by the Bradford assay (Biorad) using bovine serum albumin as a standard. Equal amounts of protein (typically 10-30 μg) are separated on a 12% SDS-PAGE gel and transferred electrophoretically to a polyvinylidene difluoride (PVDF) membrane (Hybond-P, Amersham) through Semidry-blotting (TE 70, Amersham). The membrane is blocked for 1 hour at room temperature in blocking solution [5% milk in TBS-T (25 mM Tris-HCl pH 7.4, 137 mM NaCl, 3 mM KCl, comprising 0.1% Tween-20)] and incubated with the primary antibody solution (prepared in TBS-T/1% milk) at 4° C. overnight with agitation. Antibodies specific for the following antigen is used: HO-1 (dilution 1:1000; Stress Gene) which cross reacts with constitutively expressed isoform HO-2 (36 kDa), and β-actin (1:5000, Sigma). After removal of the primary antibody solution and several washes in TBS-T, the membrane is incubated with a HRP (horseradish peroxidase)-conjugated secondary antibody (rabbit anti-mouse, 1:1000; Dako) for one hour at room temperature. Following several washes in TBS-T, detection is performed through chemiluminiscence (ECL, Amersham) and exposing to x-ray film (FIG. 7). The intensities of the bands can be analysed densitometrically using ChemiImager 5500 software (Alpha Innotech) and each signal normalised to the intensity of the corresponding HO-2 (showing 7 fold reduction of HO-1 upon MitoQ treatment when compared to DDC intoxicated group of animals), or alternatively to β-actin.

A marked decrease of DDC induced overexpression of the hemoxygenase 1 under MitoQ treatment (FIG. 7) suggests that oxidative stress is greatly reduced by antioxidants according to the invention.

In long term experiments there can be evaluated the protein expression level of cytokeratin 8 known to be increased in mice during DDC intoxication (Stumptner C. et al., 2001, Journal of Hepatology, 34: 665-675) and/or catalase reported to be reduced in (N)ASH patients (Videla L. A. et al., 2004, Clinical Science, 106: 261-268).

The protein expression level(s) of fatty acid binding protein (FABP) representing a sensitive marker for hepatocyte damage (Monbaliu D . et al., 2005, Transplant Proc. 37(1):413-416) is determined. Western blot analysis shows a significant decrease of FABP protein in DDC intoxicated mice when compared to normal mice. Furthermore, under MitoQ treatment of DDC intoxicated animals FABP reaches almost control mice FABP protein expression values (controls represent non intoxicated group of animals treated with vehicle only, see Example 3), thus suggesting the effect of MitoQ in treatment or prophylaxis of diseases according the invention.

The amount of apoptotic cells in cryostat sections derived from DDC intoxicated mice treated with the antioxidants according to the invention can be semi quantified by anti caspase 3 immunohistochemical standard methods known in prior art (Brekken et al., 2003, The Journal of Clinical Investigation, 111, 4: 487-495) and compared to appropriate controls.

In addition, protoporphyrin levels in homogenates of DDC intoxicated mice treated with the antioxidants can be determined by using fluorescence assays (Stumptner C. et al., 2001, Journal of Hepatology, 34: 665-675) and compared to appropriate controls.

### Example 6

### Evaluation of the Effect of Antioxidants According to the Inventions on Blood Parameters

Serum levels of liver specific enzymes are monitored in the Actitest (Biopredictive, Houilles, France) that provides a measure of liver damage according to the invention. The serum levels of a₂-macroglobulin, haptoglobin, γ-glutamyl transpeptidase, total bilimbin, apolipoprotein A1 and alanine aminotransferase are measured from DDC- or GF intoxicated, control, and corresponding DDC- or GF exposed animals also treated with the targeted antioxidants using the methods described in Poynard, et al., 2003, Hepatology 38:481-492, following the general time line strategy according to Example 3.

Actitest performed also with human serum as a measure of liver damage, especially fibrosis, can be similarly employed to monitor the effect of treatment of patients with these diseases with antioxidants according to the invention.

In serum from various tested animal groups following parameters indicating liver damage, namely bilirubin, alanine aminotransferase (ALT/GPT), aspartate aminotransferase (ASAT/GOT) and glutamate dehydrogenase (GLDH) are determined according to standard protocols in clinical diagnostics employing commercially available kits (No: 11552414; 11876805216; 11876848216; 11929992 all purchased by Roche AG, Switzerland) on a Hitachi/Roche 917 Analyser.

The reduction of serum liver enzymes in animals (as e.g. alanine- and aspartate aminotransferase, see FIG. 8) treated with the compounds according to the invention indicates the reduction of liver damage in such treated samples and provides support for the therapeutic efficacy of these compounds in diseases according to the invention.

### Example 7

### Measurement of Reactive Oxygen Species (ROS) in Tissue Sections

To detect in situ generation of ROS in liver specimens from DDC- or GF intoxicated and control tissues, fluorescence photomicroscopy with dihydroethidium (DHE, Molecular Probes) is performed according to standard protocols (e.g. Brandes R. P. et al., 2002, Free Radic Biol Med; 32 (11): 1116-1122). DHE is freely permeable to cells and in the presence of O₂ is oxidized to ethidium, where it is trapped in the nucleus by intercalating with the DNA. Ethidium is excited at 488 nm with an emission spectrum of 610 nm.

Liver samples are embedded in OTC Tissue Tek (Sakura Finetek Europe, Zoeterwonde, Netherlands) and frozen using liquid nitrogen-cooled isopentane. Samples are then cut into sections (5 μm-30 μm) and placed on glass slides. Dihydroethidium (5-20 μmol/L) is applied to each tissue section. The slides are subsequently incubated in a light-protected humidified chamber at 37° C. for 30 minutes and washed (2-3 times) with buffered saline solution (PBS) at 37° C. The sections are then to be coverslipped. The image of DHE is obtained by using fluorescence microscopy or laser scanning confocal imaging with a 585 nm long-pass filter.

Another approach well established in the art allows measuring the ROS production in DDC- or GF intoxicated versus control liver tissue using a lucigenin chemiluminescence assay (Goerlach A. et al., 2000, Circ Res., 87(1): 26-32). Specimens of liver tissue are equilibrated in vials containing 1 ml of 50 mmol/L HEPES (pH 7.4), 135 mmol/L NaCl, 1 mmol/L CaCl₂, 1 mmol/L MgCl₂, 5 mmol/L KCl, 5.5 mmol/L glucose, and 5 μmol/L lucigenin as the electron acceptor. The light reaction between superoxide and lucigenin is detected using a chemiluminescence reader. The chemiluminescence signal is expressed as average counts per minute per mg dry tissue measured over a 15-30 min period. The chemiluminescent signal data are revealed after subtracting the background chemiluminescence observed in the absence of specimens.

This approach enables demonstration of the elevation of ROS in the liver derived from DDC- or GF intoxicated mice thus mimicking observations made in the patients suffering from the diseases according to the invention.

### Example 8

### The Effect of Antioxidants According to the Invention on Reduction of Reactive Oxygen Species (Oxidative Damage) in Mice Exposed to DDC or GF

The general strategy of timelines and dosage regime(s) for DDC- or GF intoxication of tested animals and for their treatment with the antioxidants is identical to the experimental set-up used for determination of morphologic abnormalities (see Example 3).

The application of the antioxidants according to the invention, e.g. derivatives of vitamin E, coenzyme Q₁₀ or a glutathione peroxidase mimetic, provides a significant reduction of ROS levels in liver(s) exposed to DDC or GF. This result further implicates impact of ROS in liver damage and demonstrates that this damage is mitigated by targeting e.g. MitoQ/MitoS or MitoVit E to the mitochondria, a major cellular source of ROS. The reduction in the level of ROS measured with the methods according to Example 7 upon treatment with the targeted antioxidants indicates the therapeutic efficacy of these compounds for the diseases according to the invention.

### Example 9

### Measurement of Reactive Oxygen Species (ROS) in Liver Cell Lines

Another simple set of experiments employing hepatoma cell lines (e.g. HepG2 or Hep3B), the SNU-398 hepatocellular carcinoma-derived cell line (ATCC No. CRL-2233, LGC Promochem, Germany), the HUH-7 human carcinoma-derived cell line (Japanese collection of Research Biosources JCRB 0403) or the Tib-73 mouse embryonic liver cell line (ATCC TIB 73=BNL CL2 derived from BAL/c mouse, MD, USA) allows measurement of ROS production in these cells upon DDC intoxication. A glutathione synthesis inhibitor L-buthionine-(S,R)-sulfoximine (BSO) is employed as an alternative to elevate endogenous oxidative stress (Kito M. et al., 2002, Biochem Biophys Res Commun., 291(4): 861-867).

Since CoCl₂ has recently been shown to affect mitochondria (Jung J Y and Kim W J., 2004, Neurosci Lett., 371:85-90) in order to measure ROS production in differentiated cell lines, HepG2 are alternatively stimulated by 100 μM CoCl₂ (Sigma) (Bel Aiba R S, et al., 2004, Biol Chem. 385:249-57).

Another approach well established on cultured cells (as well as in isolated cell organelles or the entire tissue) allows measurement of the ROS production induced by antimycin A (FIG. 10) according to Chem Biol Interact. 2000 Jul. 14; 127(3):201-217, or by rotenone using lucigenin chemiluminescence assay (Goerlach A. et al., 2000, Circ Res., 87(1): 26-32).

To determine ROS production in for example hepatoma cell lines a standard experimental protocol according to Example 8 is applied. Tested hepatoma cells are grown in 96-well plates in culture medium (DMEM supplemented with 10% FCS, Gibco) to 80% confluency, subsequently washed with HBSS and incubated in the dark with DHE (10-50 μM) for 10 minutes at 37° C. Cells are then washed twice with Hank's balanced salt solution (HBSS, Gibco) to remove excess dye. Fluorescence is monitored in a fluorescence microscope (Olympus, Hamburg, Germany).

Alternatively, the generation of ROS is measured by using the fluoroprobe 5-(and-6)-chloromethyl-2′,7′-dichlorodihydrofluorescein diacetate acetyl ester (CM-H₂DCFDA, Molecular Probes, Goettingen, Germany) which is converted to fluorescent dichlorofluorescien (DCF; Djordjevic T. et al., 2004, Antioxidants & Redox Signaling, 6: 713-720). To determine DCF fluorescence in a microplate reader (Tecan, Crailsheim, Germany), cells (e.g. HepG2, Hep3B or SNU-398) are grown in 96-well plates to 80% confluency. The cells are then washed twice with Hank's balanced salt solution (HBSS, Gibco) and incubated in the dark with CM-H₂DCFDA (8.5 μM) dissolved in HBSS containing N-ω-nitro-L-arginine methyl ester (L-NAME, 10 μM) for 10 minutes at 37° C. to prevent the formation of NO. After several washes with HBSS to remove excess dye, fluorescence is monitored by using 480 nm excitation and 540 nm emission wavelength. DCF fluorescence is standardized to the number of viable cells using the Alamar Blue test according to the manufacturer's instructions (Biosource, Nivelles, Belgium). Briefly, cells are incubated with Alamar Blue in phosphate-buffered saline (PBS), pH 7.4 at 37° C. to allow the indicator to change from the oxidized (blue) to the fully reduced (red) form. The absorbance is then measured at the wavelength of 580 μm.

Optionally, ROS production is assessed by flow cytometric analysis of CM-H₂DCFDA stained cells. The cells are detached and harvested by trypsinisation, collected by centrifugation and resuspended in HBSS at a concentration of 1×10⁶ cells/ml. Cells are then loaded with 8.5 μM CM-H₂DCFDA for 15 minutes in the dark at 37° C. before stimulation. The DCF fluorescence is monitored by analyzing 10,000 cells using 480 nm excitation and 540 nm emission wavelengths in a flow cytometer (Partec, Muenster, Germany).

The hepatoma cell lines incubated for up to 72 hours in culture medium (DMEM and 10% FCS, Gibco) supplemented with DDC (EC₅₀=50 μg/ml of medium) or with up to 100 μM BSO are another suitable in vitro model mimicking observations made in patients suffering from the diseases according to the invention.

### Example 10

### The Effect of the Antioxidants According to the Invention on Reduction of Oxidative Damage in DDC-, BSO-, Antimycin A- (or Rotenone-) Intoxicated or CoCl₂ Induced Cultured Cells

By employing standard protocols and following general strategy of time lines according to Example 9, the human cell lines intoxicated with DDC or BSO, respectively, and simultaneously treated with MitoQ/MitoS or MitoVit E (in concentrations corresponding to EC₅₀=0.51 nM for MitoQ and EC₅₀=416 nM for MitoVit E according to Jauslin M. L. et al., 2003, FASEB J., 2003, (13): 1972-1974) provide a significant reduction in ROS formation.

In another experiment HepG2 stimulated by 100 μM CoCl₂ (Sigma) are used (Bel Aiba R. S. et al., 2004,. Biol Chem. 385:249-57). Following the experimental set up described in Example 9, HepG2 cells are plated on a 96-well plate and serum starved for 16 h prior to the experiment. HepG2 are then washed once with HBSS (Hanks' Balanced Salt Solution, Gibco) and incubated with MitoQ in concentration range of 0.5 to 10 μM or the respective amount of DMSO (Sigma). After 15 min DCF is added to the cells (final concentration of 8 μM) and cells are incubated with the dye for 10 min. After loading the media is removed and fresh HBSS is added containing MitoQ and CoCl₂ (100 μM). The fluorescence is measured in a plate-reader (Tecan Safire) after 0, 10, 20 and 30 minutes (Djordjevic T, et al., 2005, Free Radic Biol Med. 38:616-30).

Already unstimulated cells treated with 5 μM MitoQ show reduce basal ROS production. CoCl₂-stimulated ROS production (100 μM CoCl₂) is significantly decreased by 5 μM MitoQ suggesting that antioxidants according to the invention significantly decrease basal and CoCl₂-stimulated ROS levels in these cells (FIG. 9).

Another approach allows measurement of the ROS production induced by antimycin A or rotenone by using lucigenin chemiluminescence assay (experimental set up as in Example 9) in e.g. HUH-7 or Tib-73. HUH-7 cells are incubated in 6 well plates and stimulated by using antimycin A in concentration 0-25 μM (preferably 0, 1 and 5 μM) simultaneously with or without MitoQ (or MitoS) in concentration range from 0 to 1000 nmol dissolved in DMEM (Gibco) for 3 hours at 37° C. After 3 subsequent washings the cells are equilibrated in plates containing 1 ml of 50 mmol/L HEPES (pH 7.4), 135 mmol/L NaCl, 1 mmol/L CaCl₂, 1 mmol/L MgCl₂, 5 mmol/L KCl, 5.5 mmol/L glucose, and 5 μmol/L lucigenin as the electron acceptor. The light reaction between superoxide and lucigenin is detected using a chemiluminescence reader (Lumistar, BMG laboratories, Germany). The chemiluminescence signal is expressed as average counts per minute and normalized to cell number as determined by cell counter (Casy Technology Instrument, Schärfe-System, Germany).

Overall, these experiments show a significant reduction in ROS formation (FIG. 10), thus further confirming a therapeutic benefit of mitochondrially targeted antioxidants in liver disorders according to the invention.

### Example 11

### Evaluation of Effect of Antioxidant Compounds on Nude Mice

The general strategy to determine the effect(s) of mitochondrially targeted antioxidants according to the invention in treatment and/or prevention of epithelial cancers follows the treatment paradigms described above for DDC- or GF intoxicated mice (according to Examples 2 to 7) but instead employs immunocompromised mice harbouring human epithelial cell cancer xenografts (nude mice tumor xenografts applied to e.g. CD1 nu/nu mice from Charles Rivers Laboratories, USA). Tumor cell lines or primary tumors that are xenografted subcutaneously according to standard methods (Li K. et al., 2003, Cancer Res., 63(13): 3593-3597) include colon adenocarcinomas, invasive ductal carcinomas of the breast, small and non-small cell lung carcinoma, prostate tumors, pancreatic tumors and stomach tumors.

Tumor-derived cell lines (grown in DMEM/10% FBS) are harvested in log-phase growth, washed twice with PBS, resuspended in 1 ml PBS (2.5×10⁷ cells/ml), and injected subcutaneously into the right flank of a nude mouse (Hsd: athymic nu/nu, Harlan Winkelmann; aged between 5 and 6 weeks) at 5×10⁶ cells/mouse (0.2 ml). Tumor growth is monitored every other day for the indicated periods (depending on the cell type). Tumor size is determined by the product of two perpendicular diameters and the height above the skin surface.

Treatment of such mice with e.g. MitoQ (MitoS) demonstrates reduced growth of tumors, increased necrosis of the tumors and decreased vascularization of the tumor xenografts. Similarly, the levels of ROS in nude mice tumor xenografts are monitored as described above and are reduced in xenograft tumors treated with the antioxidants according to the invention.

It will be apparent to those skilled in the art that various modifications can be made to the compositions and processes of this invention. Thus, it is intended that the present invention cover such modifications and variations, provided they come within the scope of the appended claims and their equivalents. All publications cited herein are incorporated in their entireties by reference.

1. A method of treating a patient with actual or expected liver disease or epithelial cancer which comprises administering to the patient in need thereof a therapeutically or prophylactically effective amount of a mitochondrially targeted antioxidant compound comprising a lipophilic cation covalently coupled to an antioxidant moiety.

2. The method according to claim 1 wherein the liphophilic cation is the triphenylphosphonium cation.

3. The method according to claim 1 wherein the compound has the formula 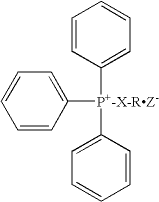
wherein X is a linking group, Z⁻ is an anion and R is an antioxidant moiety.

4. The method according to claim 3 wherein the antioxidant moiety R is a quinone or a quinol.

5. The method according to claim 4 wherein the compound has the formula

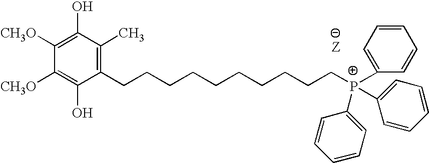

6. The method according to claim 3 wherein the antioxidant moiety R is a glutathione peroxidase mimetic.

7. The method according to claim 6 wherein the glutathione peroxidase mimetic moiety is

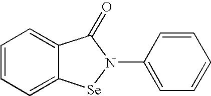

8. The method according to claim 3 wherein the antioxidant moiety R is selected from the group consisting of vitamin E and vitamin E derivatives, chain breaking antioxidants, including butylated hydroxyanisole, butylated-hydroxytoulene, general radical scavengers including derivatised fullerenes, spin traps including derivatives of 5,5-dimethylpyrroline N-oxide, tert-butylnitrosobenzene, and α-phenyl-tert-butylnitrone.

9. The method according to claim 3 wherein the antioxidant moiety R is vitamin E or a vitamin E derivative.

10. The method according to claim 9 wherein the compound has the formula

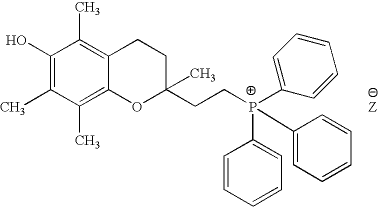

11. The method according to claim 3 wherein the antioxidant moiety R is butylated hydroxyanisole or butylated hydroxytoulene.

12. The method according to claim 3 wherein the antioxidant moiety R is a derivatised fullerene.

13. The method according to claim 3 wherein the antioxidant moiety R is a 5,5-dimethylpyrroline N-oxide, tert-butylnitrosobenzene, α-phenyl-tert-butylnitrone and derivatives thereof.

14. The method according to claim 13 wherein the compound has the formula

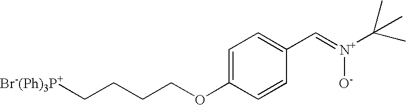

15. The method according to claim 3 wherein the linking group X is a C₁ to C₃₀ carbon chain, optionally including one or more double or triple bonds, and optionally including one or more unsubstituted or substituted alkyl, alkenyl or alkynyl side chains.

16. The method according to claim 15 wherein the linking group X is (CH₂)ₙ where n is an integer from 1 to 20.

17. The method according to claim 16 wherein the linking group X is an ethylene, propylene, butylene, pentylene or decylene group.

18. The method according to claim 3 wherein the anion Z⁻ is a pharmaceutically acceptable anion.

19. The method according to claim 18 wherein Z⁻ is halide.

20. The method according to claim 19 wherein Z⁻ is bromide.

21. The method according to claim 18 wherein Z⁻ is the anion of an alkane- or arylsulfonic acid.

22. The method according to claim 21 wherein Z⁻ is methanesulfonate.

23. The method according to claim 22 wherein the compound has the formula

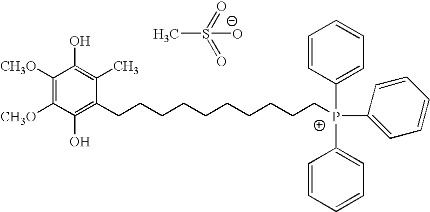

24. The method according to claim 1, wherein the liver disease is a disease selected from the group consisting of alcoholic liver disease, non-alcoholic fatty liver disease, steatosis, cholestasis, liver cirrhosis, nutrition-mediated liver injury, toxic liver injury, infectious liver disease, liver injury in sepsis, autoimmune-mediated liver disease, hemochromatosis, alphal antitrypsin deficiency, radiation-mediated liver injury, liver cancer, benign liver neoplasms and focal nodular hyperplasia.

25. The method according to claim 1, wherein the liver disease is a disease selected from the group consisting of alcoholic liver disease, non-alcoholic fatty liver disease, steatosis, cholestasis, liver cirrhosis, nutrition-mediated liver injury, toxic liver injury, infectious liver disease, liver injury in sepsis, autoimmune-mediated liver disease, hemochromatosis, alphal antitrypsin deficiency and radiation-mediated liver injury.

26. The method according to claim 1 wherein the liver disease is alcoholic liver disease or non-alcoholic fatty liver disease.

27. The method according to claim 1 wherein the liver disease is alcoholic steatohepatitis or non-alcoholic steatohepatitis.

28. The method according to claim 1 wherein the liver disease is alcoholic steatohepatitis.

29. The method according to claim 1 wherein the liver disease is non-alcoholic steatohepatitis.

30-31. (canceled)

32. The method according to claim 1 wherein the liver disease is infectious liver disease.

33. The method according to claim 1 wherein the liver disease is hepatitis C.
